SEQUENCE LISTING (26 sequences)

SEQ ID NO: 1 - DNA, 23 bp, synthetic construct
  source 1..23: mol_type = other DNA
tgtgcaaatctatgcaaaactga

SEQ ID NO: 2 - DNA, 22 bp, synthetic construct
  source 1..22: mol_type = other DNA
tagcttatcagactgatgttga

SEQ ID NO: 3 - DNA, 21 bp, synthetic construct
  source 1..21: mol_type = other DNA
atcacattgccagggatttcc

SEQ ID NO: 4 - DNA, 22 bp, synthetic construct
  source 1..22: mol_type = other DNA
tattgcacttgtcccggcctgt

SEQ ID NO: 5 - DNA, 22 bp, synthetic construct
  source 1..22: mol_type = other DNA
cacccgtagaaccgaccttgcg

SEQ ID NO: 6 - DNA, 22 bp, synthetic construct
  source 1..22: mol_type = other DNA
acaggtgaggttcttgggagcc

SEQ ID NO: 7 - DNA, 22 bp, synthetic construct
  source 1..22: mol_type = other DNA
taacactgtctggtaaagatgg

SEQ ID NO: 8 - DNA, 22 bp, synthetic construct
  source 1..22: mol_type = other DNA
tctcccaacccttgtaccagtg

SEQ ID NO: 9 - DNA, 24 bp, synthetic construct
  source 1..24: mol_type = other DNA
tttggcaatggtagaactcacact

SEQ ID NO: 10 - DNA, 22 bp, synthetic construct
  source 1..22: mol_type = other DNA
taatactgcctggtaatgatga

SEQ ID NO: 11 - DNA, 22 bp, synthetic construct
  source 1..22: mol_type = other DNA
tgtcagtttgtcaaatacccca

SEQ ID NO: 12 - DNA, 19 bp, synthetic construct
  source 1..19: mol_type = other DNA
aatggatttttggagcagg

SEQ ID NO: 13 - DNA, 17 bp, synthetic construct
  source 1..17: mol_type = other DNA
ctcgcttcggcagcaca

SEQ ID NO: 14 - DNA, 20 bp, synthetic construct
  source 1..20: mol_type = other DNA
aacgcttcacgaatttgcgt

SEQ ID NO: 15 - DNA, 20 bp, synthetic construct
  source 1..20: mol_type = other DNA
gtagttctggggcgtgttca

SEQ ID NO: 16 - DNA, 20 bp, synthetic construct
  source 1..20: mol_type = other DNA
taggtggatggggagcttca

SEQ ID NO: 17 - DNA, 23 bp, synthetic construct
  source 1..23: mol_type = other DNA
gccagaaaatactgatgttcact

SEQ ID NO: 18 - DNA, 17 bp, synthetic construct
  source 1..17: mol_type = other DNA
tcggtcggccaacaacc

SEQ ID NO: 19 - DNA, 20 bp, synthetic construct
  source 1..20: mol_type = other DNA
gtgaaggtggcagaaggaca

SEQ ID NO: 20 - DNA, 20 bp, synthetic construct
  source 1..20: mol_type = other DNA
tgggttgttcactcccacag

SEQ ID NO: 21 - DNA, 20 bp, synthetic construct
  source 1..20: mol_type = other DNA
accagcatccattgcaaacc

SEQ ID NO: 22 - DNA, 20 bp, synthetic construct
  source 1..20: mol_type = other DNA
tggtaccaaccgcacaaaca

SEQ ID NO: 23 - DNA, 22 bp, synthetic construct
  source 1..22: mol_type = other DNA
cctggaagaatgtgtcacgcta

SEQ ID NO: 24 - DNA, 22 bp, synthetic construct
  source 1..22: mol_type = other DNA
gctttctctcttcttctgcctg

SEQ ID NO: 25 - DNA, 21 bp, synthetic construct
  source 1..21: mol_type = other DNA
tatcgtgatgctagtccgatg

SEQ ID NO: 26 - DNA, 22 bp, synthetic construct
  source 1..22: mol_type = other DNA
tgcagctagctgcatcgatcgg

(19) United States
(12) Patent Application Publication
(10) Pub. No.: US 2025/0065298 A1
(43) Pub. Date: Feb. 27, 2025

(54) Composition for separating extracellular vesicles comprising amine-functionalized solid support and homobifunctional hydrazide

(71) Applicant: UIF (University Industry Foundation), Yonsei University, Seoul (KR)

(72) Inventors: Yong SHIN, Seoul (KR); Bonhan KOO, Gunpo-si (KR)

(21) Appl. No.: 18/809,675

(22) Filed: Aug. 20, 2024

(30) Foreign Application Priority Data
Aug. 22, 2023 (KR) 10-2023-0110156
Aug. 2, 2024 (KR) 10-2024-0102971

(51) Int. Cl. B01J 20/16; B01J 20/22; B01J 20/28; C12N 15/10

(52) U.S. Cl. CPC B01J 20/165; B01J 20/22; B01J 20/28004; C12N 15/1017; B01J 2220/64

(57) ABSTRACT

Proposed are an extracellular vehicle-isolating composition including a homobifunctional hydrazide and a solid support functionalized with amine groups, and a method of isolating extracellular vesicles by using the composition. The method may isolate highly enriched high-purity EVs from a biological sample and also enable simultaneous operation for EVs isolation and extraction of EV-derived proteins or EV-derived nucleic acids. The method is low-cost and simple, does not require special equipment other than a syringe filter, and allows for high-throughput experiments due to the use of large-volume samples. The method may reduce the time required for protein and nucleic acid extraction by simplifying the experiments. Additionally, this EV enrichment may increase the sensitivity of biomarker detection for disease diagnosis and treatment.

### CROSS REFERENCE TO RELATED APPLICATION

The present application claims priority to Korean Patent Application No. 10-2023-0110156, filed Aug. 22, 2023, the entire contents of which is incorporated herein for all purposes by this reference.

### INCORPORATION BY REFERENCE OF SEQUENCE LISTING XML

The present application contains a Sequence Listing file named: “23884-3 Sequence Listing XML”, Size: 24,099 bytes, Date of Creation: Oct. 21, 2024, which has been submitted in XML format via Patent Center, the entirety of which is incorporated herein by specific reference.

### BACKGROUND OF THE DISCLOSURE

### 1. Field of the Disclosure

The present disclosure relates to an extracellular vesicle-isolating composition including a homobifunctional hydrazide and a solid support functionalized with an amine group and to a method of isolating extracellular vesicles by using the same.

### 2. Description of the Related Art

Recently, the importance of small extracellular vesicles (EVs, <200 nm) has greatly increased in the diagnosis of cancer, including colorectal cancer (CRC). EVs are present in cell culture media and body fluids such as plasma, serum, saliva, and urine, and EVs act as intercellular messengers that exchange biological substances between cells. Thus, EVs perform important functions in biological processes, including cellular metabolism and signaling.

EVs richly contain bioactive molecules such as proteins, nucleic acids (NAs), and lipids which reflect the physiological state of the parent cell. In particular, tumor-derived EVs released from cancer cells have emerged as promising biomarkers for early cancer detection, potentially revealing information about the presence and progression of cancer. Among the cargo of EVs, non-coding RNAs such as EV-derived circular RNAs (circRNAs) and microRNAs (miRNAs) play important roles in various cellular functions and are involved in several pathological processes, so the RNAs have attracted considerable research interest. The stability of circRNAs and miRNAs in body fluids and the unique expression profiles of the circRNAs and miRNAs in cancer show the potential of the circRNAs and miRNAs as powerful biomarkers for cancer. However, the functions and mechanisms of the circRNAs and miRNAs in tumor-derived EVs are not fully understood. This gap limits the potential application of circRNAs and miRNAs in cancer diagnosis and highlights the need for further research.

EV isolation is important to obtain reliable information about detection sensitivity and accuracy of EV-derived biomarkers, which play an important role in cancer diagnosis and prognosis assessment. Accordingly, recent emphasis has been placed on the development of rapid and convenient technologies to isolate highly enriched high-purity EVs from limited samples.

An ultracentrifugation (UC) method is currently the most commonly used technique for EV isolation. The UC method isolates EVs based on size and mass at specific centrifugation speeds. Due to the standardized protocols of the UC method, consistent results are obtained. The UC method may be applied to a variety of samples and may isolate EVs at high purity and yield. However, the UC method requires expensive ultracentrifuges and is complex, labor-intensive, and time-consuming. Additionally, EV-derived biomarkers may be lost due to high-speed centrifugation-caused physical damage, lowering detection sensitivity.

An alternative to the UC method is a total exosome isolation (TEI) method, which uses polymers to form EV aggregates and isolate EVs through sedimentation. This method allows highly enriched EV isolation at lower centrifugation speeds than the UC method. However, the TEI method still requires a centrifuge device, requires overnight incubation for the polymer-EV reaction, and carries the risk of polymer additives, residual lipoproteins, and other proteins co-precipitating with EVs, potentially lowering EV purity.

Therefore, it is clear that there is an urgent need to develop a new method that may accurately, conveniently, and economically isolate highly enriched high-purity EVs.

### SUMMARY OF THE DISCLOSURE

To address the limitations, the present inventors synthesized zeolite-NH₂ to be placed on the surface of a syringe filter, the zeolite-NH₂ being functionalized with an amine group (NH₂) that imparts a positive charge for EV capture, and then the present inventors used the zeolite-NH₂ together with a homobifunctional hydrazide as a new cross-linking agent. As a result, the present inventors discovered that highly enriched high-purity EVs may be enriched and isolated from biological samples and that EV-derived proteins or EV-derived nucleic acids may be extracted simultaneously with EV isolation, thereby completing the present disclosure.

Accordingly, the present disclosure is to provide a composition for isolating extracellular vesicles (EVs) from a biological sample, the composition including a homobifunctional hydrazide and a solid support functionalized with amine groups (NH₂).

In addition, the present disclosure is also to provide a method of isolating extracellular vesicles (EVs) from biological samples by using the composition.

In addition, the present disclosure is also to provide a method of isolating extracellular vesicles (EVs) from biological samples, the method including:
(a) adding a composition to biological samples containing extracellular vesicles (EVs);
(b) transferring the resulting mixture obtained through the (a) adding into a syringe and connecting a syringe filter to the syringe;
(c) pressing the syringe to remove unreacted materials and enriching the extracellular vesicles (EVs) bound to zeolite-NH₂ on the surface of the syringe filter;
(d) isolating the extracellular vesicles (EVs) from the zeolite-NH₂ by injecting a high pH elution buffer; and
(e) recovering the isolated extracellular vesicles (EVs). However, the technical limitations to be addressed by the present disclosure are not limited to the limitations mentioned above. Other limitations not mentioned will be clearly understood by those skilled in the art from the description below. To achieve the purposes of the present disclosure as described above, in one aspect, the present disclosure provides an extracellular vehicle-isolating composition including a homobifunctional hydrazide and a solid support functionalized with amine groups (NH₂) for isolating extracellular vesicles (EVs) from biological samples.

In one embodiment of the present disclosure, the solid support may be one or more types selected from the group consisting of zeolite, silica gel, and diatomaceous earth.

In another embodiment, the homobifunctional hydrazide may have a structure of Formula I or II:

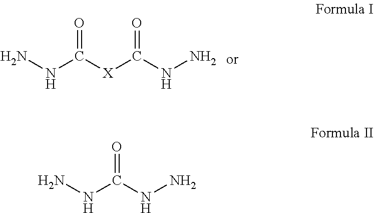

In Formula I, X is (CH₂)ₙ, and n is an integer of 0 to 10.

In yet another embodiment, n may be an integer of 0 to 4.

In yet another embodiment, the homobifunctional hydrazide may be one or more types selected from the group consisting of adipic acid dihydrazides, malonic dihydrazides, oxalyl dihydrazides, succinic dihydrazides, and carbonic dihydrazides that respectively have the following structures:

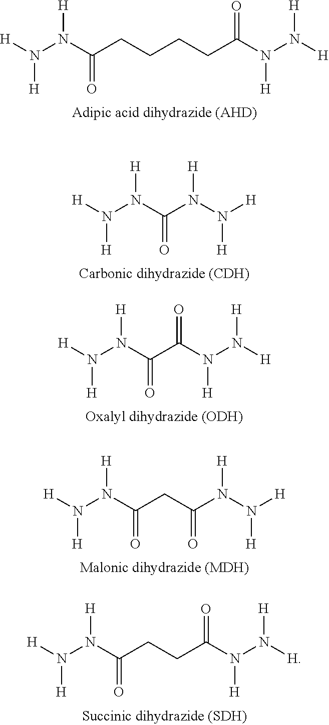

In yet another embodiment, the zeolite-NH₂ may be added at a concentration of 1 to 10 mg/ml per 1 ml of biological samples. In yet another embodiment, the zeolite-NH₂ may be added at a concentration of 2.5 to 5 mg/ml per 1 ml of biological samples. In yet another embodiment, the homobifunctional hydrazide may be added at a concentration of 1 to 100 mg/ml per 1 ml of biological samples.

In yet another embodiment, the homobifunctional hydrazide may be added at a concentration of 25 to 50 mg/ml per 1 ml of biological samples.

In yet another embodiment, the composition may be used in conjunction with a syringe filter.

In yet another embodiment, the syringe filter may have a pore size in a range of 0.22 to 3 um.

In another aspect, the present disclosure provides a method of isolating extracellular vesicles (EVs) from biological samples by using the composition.

In yet another aspect, the present disclosure provides a method of isolating extracellular vesicles (EVs) from biological samples, the method including:
(a) adding a composition to biological samples containing extracellular vesicles (EVs);
(b) transferring the resulting mixture obtained through the (a) adding into a syringe and connecting a syringe filter to the syringe;
(c) pressing the syringe to remove unreacted materials and enriching the extracellular vesicles (EVs) bound to zeolite-NH₂ on the surface of the syringe filter;
(d) isolating the extracellular vesicles (EVs) from the zeolite-NH₂ by injecting a high pH elution buffer; and
(e) recovering the isolated extracellular vesicles (EVs).

In yet another embodiment, the method may further include incubating the mixture for EV capture after the (a) adding.

In yet another embodiment, the method may further include washing the extracellular vesicles (EVs) enriched on the surface of the syringe filter by injecting phosphate-buffered saline (PBS) after the (c) pressing.

In yet another embodiment, the syringe filter used in the (b) transferring may have a pore size in a range of 0.22 to 3 μm.

In yet another embodiment, in the (d) isolating, when a RIPA lysis buffer is injected instead of the high pH elution buffer, EV-derived proteins may be extracted.

In yet another embodiment, in the (d) isolating, when (i) a non-chaotropic lysis buffer is injected instead of the high pH elution buffer, and then (ii) a high pH elution buffer is injected, EV-derived nucleic acids may be extracted.

In yet another embodiment, the non-chaotropic lysis buffer may be an NP-40 lysis buffer.

The present disclosure relates to an extracellular vehicle-isolating composition including a homobifunctional hydrazide and a solid support functionalized with amine groups and a method of isolating extracellular vesicles by using the composition. The method can isolate highly enriched high-purity EVs from a biological sample and also enable simultaneous operation for EVs isolation and extraction of EV-derived proteins or EV-derived nucleic acids. The method is low-cost and simple, does not require special equipment other than a syringe filter, and allows for high-throughput experiments due to the use of large-volume samples. The method can reduce the time required for protein and nucleic acid extraction by simplifying the experiments. Additionally, this EV enrichment can increase the sensitivity of biomarker detection for disease diagnosis and treatment.

### BRIEF DESCRIPTION OF THE DRAWINGS

FIG. 1 schematically depicts the process of isolating extracellular vesicles (EVs) from biological samples using a zeolite-NH2-homobifunctional hydrazide (HH) combination,
(1) preparation of biological samples, zeolite-NH₂ (AZ) and homobifunctional hydrazide (HH),
(2) a process of adding the zeolite-NH₂ and homofunctional hydrazide (HH) to biological samples, incubating the mixture for EV capture, transferring the incubated mixture to a syringe, and connecting a syringe filter of the syringe,
(3) enriched EVs bound to zeolite-NH₂ on the surface of the syringe filter,
(4-1) isolation of EVs using a high pH elution buffer,
(4-2) extraction of EV-derived proteins using a RIPA lysis buffer, and
(4-3) extraction of EV-derived nucleic acids using a non-chaotropic lysis buffer followed by a high pH elution buffer;

FIGS. 2A to 2E show characterization data for a zeolite-NH₂-malonic dihydrazide (MDH) combination, the MDH being one type of the homobifunctional hydrazide,
(a) SEM image of synthesized zeolite-NH₂, (b) SEM image of EVs captured by zeolite-NH₂ (highlighted in yellow) and subsequently released EVs (highlighted in red), (c) size distribution of zeolite-NH₂, (d) FT-IR spectra of zeolite-NH₂, zeolite-NH₂-MDH, zeolite-NH₂-MDH-EV, and pure MDH, and (e) zeta potential values of zeolite-NH₂, zeolite-NH₂-MDH, and zeolite-NH₂-MDH-EV;

FIGS. 3A to 3C show the results of comparing the efficiency of different types of homobifunctional hydrazides, (a, b) EV-derived protein extraction efficiencies, and (c) EV-derived nucleic acid extraction efficiency;

FIGS. 4A to 4C show optimization data of concentrations of zeolite-NH₂ and homobifunctional hydrazide (herein, the homobifunctional hydrazide is assumed to be MDH), and optimization data of syringe filter type,
(a) optimization of zeolite-NH₂ concentration (1, 2.5, 5, and 10 mg/ml), (b) optimization of MDH concentration (10, 25, 50, and 10 mg/ml), (c) optimization of syringe filter type (PTFE and PVDF) and pore size (3, 1, 0.45, and 0.22 μm), and error bars represent the standard deviation of a single experiment (N≥3);

FIGS. 5A to 5J show comparative data obtained using the zeolite-NH₂-HH combination method and conventional EV isolation methods,
(a) representative TEM image of EVs isolated by the zeolite-NH₂-HH combination method,
(b) representative TEM image of EVs isolated by an ultracentrifugation (UC) method,
(c) representative TEM image of EVs isolated by a total exosome isolation (TEI) method,
(d) representative TEM image of CD9-labeled EVs isolated by the zeolite-NH₂-HH combination method,
(e) representative TEM image of CD9-labeled EVs isolated by the ultracentrifugation (UC) method,
(f) representative TEM image of CD9-labeled EVs isolated by the total exosome isolation (TEI) method,
(g) zeta potential of isolated EVs,
(h) purity of EVs based on the ratio of EV particle concentration to protein concentration, the ratio being determined using NTA and Bradford assays,
(i) western blotting analysis for detection of EV-specific tetraspanin family proteins (CD9, CD63, and CD81),
(j) western blotting analysis for detection of non-specific protein markers (GRP78 for apoptotic bodies, GM130 for Golgi apparatus, and calnexin for endoplasmic reticulum), and error bars represent the standard deviation of a single experiment (N≥3);

FIGS. 6A to 6F show the concentration and size distribution of EVs isolated by the zeolite-NH₂-HH combination method and conventional EV isolation methods, respectively,
NTA analysis results of EVs isolated by (a) the zeolite-NH₂-HH combination method, (b) the UC method, and (c) the TEI method: determination of the concentration and size distribution based on signal intensity, and
DLS analysis results of EVs isolated by (d) the zeolite-NH₂-HH combination method, (e) the UC method, and (f) the TEI method: determination of the size distribution based on particle count;

FIG. 7 shows the results of comparing the extraction efficiency of EV-derived nucleic acids depending on the addition of additional homobifunctional hydrazide (HH) and zeolite-NH₂ (AZ) when extracting EV-derived nucleic acids; and

FIGS. 8A to 8B show the detection results of EV-derived non-coding RNA biomarkers for colorectal cancer (CRC) using the zeolite-NH₂-HH combination,
(a) qPCR analysis of twelve CRC-related miRNAs (miR-19, miR-21, miR-23, miR-92, miR-99, miR-125, miR-141, miR-150, miR-182, miR-200, miR-223, and miR-1246, in purple) and a housekeeping gene U6 (in red), and
(b) qPCR analysis of five CRC-related circRNA markers (CircLPAR1, CircPACRGL, CircLONP2, CircERBIN, and CircPNN, in orange) and a housekeeping gene GAPDH (in red).

FIGS. 9A to 9I shows clinical validation of biomarkers identified in cell line models using blood plasma samples with the zeolite-NH₂-HH-based extracellular vesicle isolation (ZAHVIS) platform,
(a) schematic overview of using clinical samples to validate biomarkers identified in cell line models.
(b to e) relative quantification (RQ) values of clinically validated miRNAs in CRC patients and healthy control (HC) individuals including (b) miR-23a-3p, (c) miR-92a-3p, (d) miR-125a-3p, and (e) miR-150-5p,
(f) CEA levels in CRC patients and HC individuals,
(g) t-Distributed stochastic neighbor embedding (t-SNE) visualization of combined biomarkers discriminating between CRC patients and HC individuals,
(h) receiver operating characteristic (ROC) curves for single biomarkers illustrating their diagnostic performance with corresponding area under the curve (AUC) values,
(i) performance evaluation of single biomarkers using Youden's index, including sensitivity, specificity, accuracy, and F1 score.

FIGS. 10A to 10M shows AI-driven analysis of blood biomarker combinations for CRC in the ZAHV-AI system,
(a) schematically depicts the ZAHV-AI system workflow for evaluating biomarker combinations,
(b to f) ROC curves showing diagnostic performance of (b) single markers, (c) 2-marker combinations, (d) 3-marker combinations, (e) 4-marker combinations, and (f) a 5-marker combination,
(g to k) AUC values for biomarker combinations, ranked by performance, including 2-marker (g) single markers, (h) combinations, (i) 3-marker combinations, (j) 4-marker combinations, and (k) a 5-marker combination,
(l) performance evaluation of all biomarker combinations using Youden's index, including sensitivity, specificity, accuracy, and F1 score,
(m) bar chart showing performance metrics for the optimal combination (miR-23a-3p, miR-92a-3p, miR-150-5p, and carcinoembryonic antigen (CEA)).

FIGS. 11A to 11B shows optimal combinations and diagnostic performance for overall and stage-specific CRC diagnosis in the ZAHV-AI system,
(a) schematic overview of the optimal blood biomarker combinations identified for overall CRC, early-stage CRC (stages 0-2), and advanced-stage CRC (stages 3-4),
(b) diagnostic performance metrics (AUC, sensitivity, specificity, accuracy, F1 score) for the optimal combinations, highlighting their effectiveness in CRC diagnosis across overall CRC, early-stage CRC, advanced-stage CRC, and individual CRC stages (stages 0-1, stage 2, stage 3, and stage 4).

### DESCRIPTION OF THE PREFERRED EMBODIMENTS

Hereinafter, the present disclosure will be described in detail.

The present disclosure provides an extracellular vehicle-isolating composition including a homobifunctional hydrazide (HH) and a solid support functionalized with amine groups.

In the present disclosure, all solid materials commonly used as adsorbents in the art may be used as the solid support. Representative solid supports may include zeolite and silica-based materials such as silica gel or diatomaceous earth. In one embodiment, the solid support may be one or more types selected from the group consisting of zeolite, silica gel, and diatomaceous earth but is not limited thereto.

Methods of functionalizing a solid support with amine groups are known in the art. A representative method is to functionalize hydroxyl groups with amine groups using silanization. Materials such as 3-minopropyl(diethoxy)methylsilane (APDMS) and (3-aminopropyl)triethoxysilane (APTES) may be used.

FIG. 1 schematically shows a process for efficiently and rapidly isolating extracellular vesicles (EVs) from biological samples by using zeolite-NH₂ and a homobifunctional hydrazide as functional materials for a solid support functionalized with amine groups according to an embodiment.

First, the present inventors synthesized zeolite-NH₂ as a solid support being functionalized with amine groups (NH₂) that impart a positive charge to the surface of zeolite for EV capture. Zeolites are efficient in isolating and preserving biological molecules due to the zeolites' selective adsorption of specific molecules, high adsorption capacity, chemical and thermal stability, reusability, and environmental friendliness. Zeolites also have a large surface area, uniform pore size, and excellent ion exchangeability, so zeolites may efficiently capture negatively charged EVs. Accordingly, the advantages of zeolite use are further growing.

In the present disclosure, homobifunctional hydrazides are used with the zeolite-NH₂ as a new cross-linking agent. The homobifunctional hydrazides have highly reactive and chemically tunable structures, so the homobifunctional hydrazides play an important role in the present disclosure by greatly improving the efficiency and precision of EV capture.

The term “extracellular vesicle”, used herein, is used interchangeably with the English abbreviation “EV”.

The term “biological sample”, used herein, is a broad concept that includes all types of specimens derived from humans or animals. Specifically, biological samples include blood, serum, plasma, saliva, urine, cerebrospinal fluid, gastric secretions, mucous membrane samples, peritoneal samples, nasal secretions, sputum, and pharyngeal exudate but is not limited thereto. Cell-free supernatants used in the embodiments described below are also included within the scope of the biological samples.

The term “zeolite-NH₂”, used herein, refers to zeolite functionalized with amine groups (NH₂) that impart a positive charge to the surface of the zeolite. The term, herein, is used interchangeably with “amine-functionalized zeolite” or its English abbreviation “AZ”.

The term “homobifunctional hydrazide”, used herein, refers to a compound that provides dual functionality by having each hydrazide reactive group (C═ONHNH₂) at both ends of the molecule. The term, herein, is used interchangeably with the English abbreviation “HH”.

In another embodiment, the homobifunctional hydrazide may have a structure of Formula I or II:

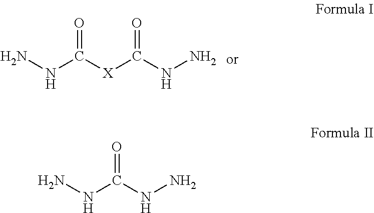

In Formula I, X is (CH₂)ₙ, and n is an integer of 0 to 10.

Specifically, n may be an integer of 0 to 4, and more specifically, n may be 0, 1, 2, 3, or 4.

In yet another embodiment, the homobifunctional hydrazide may be one or more types selected from the group consisting of adipic acid dihydrazide, malonic dihydrazide, oxalyl dihydrazide, succinic dihydrazide, and carbonic dihydrazide but is not limited thereto.

In yet another embodiment, the homobifunctional hydrazide may be adipic acid dihydrazide (ADH), which is a compound with a structure of Formula (I) in which n is 4.

In yet another embodiment, the homobifunctional hydrazide may be malonic dihydrazide (MDH), which is a compound with a structure of Formula (I) in which n is 1.

In yet another embodiment, the homobifunctional hydrazide may be oxalyl dihydrazide (ODH), which is a compound with a structure of Formula (I) in which n is 0.

In yet another embodiment, the homobifunctional hydrazide may be succinic dihydrazide (SDH), which is a compound with a structure of Formula (I) in which n is 2.

In yet another embodiment, the homobifunctional hydrazide may be carbonic dihydrazide (CDH), which is a compound with a structure of Formula (II).

ADH, CDH, MDH, ODH, and SDH respectively have the following structures.

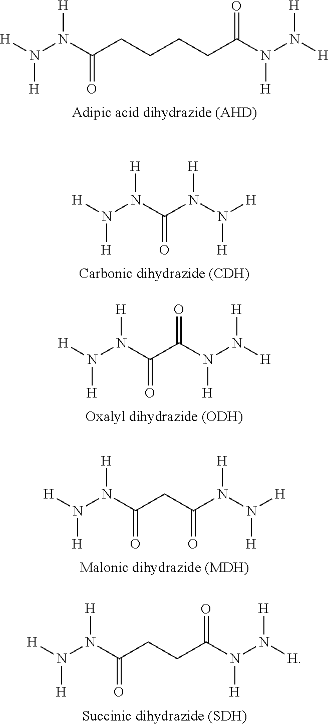

One of the key properties of the homobifunctional hydrazide is having each hydrazide reactive group (C═ONHNH₂) at both ends of the molecule, thereby the different types of the homobifunctional hydrazide provide dual functionality. Hydrazine groups (NH-NH₂) inherently carry a positive charge, allowing electrostatic interaction with negatively charged EVs.

Additionally, the carbonyl components (C—O) in the hydrazide groups may form imine bonds with the amine of zeolite-NH₂, resulting in amplified bonding force between zeolite-NH₂ and EVs. This multiplicative and robust binding mechanism between the zeolite-NH₂, homobifunctional hydrazides, and EVs not only facilitates the successful EV capture and safe binding of EVs to the zeolite-NH₂ surface but also achieves efficient enrichment in a remarkably swift span of only 10 minutes.

In yet another embodiment, the zeolite-NH₂ may be added at a concentration of 1 to 10 mg/ml per 1 ml of biological samples. More specifically, the zeolite-NH₂ may be added at a concentration of 2.5 to 5 mg/ml per 1 ml of biological samples.

In yet another embodiment, the homobifunctional hydrazide may be added at a concentration of 1 to 100 mg/ml per 1 ml of biological samples. More specifically, the homobifunctional hydrazide may be added at a concentration of 25 to 50 mg/ml per 1 ml of biological samples.

After EVs are effectively attached to zeolite-NH₂, the zeolite-NH₂-EVs undergo a syringe filtration process. This process is crucial as the zeolite-NH₂ particles are larger than the pore size of the syringe filters, enabling selective filtration. In this process, unbound molecules other than the EVs bound to zeolite-NH₂ pass through the filters and are discarded as waste, and the EVs are enriched on the filter surface. The ease of use, speed, and precision of the syringe filters combined with the zeolite-NH₂ and homobifunctional hydrazide facilitate sample processing and significantly reduce the time required for EV isolation.

Accordingly, the composition of the present disclosure may be used by using the syringe filters. It is sufficient when the pore size of the syringe filters is smaller than the solid support used. In other words, it is sufficient when the solid support is larger than the pore size of the syringe filters so that the solid support does not pass through the syringe filters and remains on the syringe filter surface. Those skilled in the art will understand that the pore size of the syringe filters may vary depending on the type of syringe filter. In the embodiments described later, commercially available PVDF and PTFE filters with a pore size diameter of 3, 1, 0.45, and 0.22 μm were used.

However, syringe filters with various pore sizes may be used depending on the various types of filters and the size of solid supports.

In yet another embodiment, the syringe filters may have a pore size of 0.22 to 3 um, and more specifically, the syringe filters may have a pore size of 0.22 to 0.45 μm.

The combination of the solid support functionalized with amine groups and homobifunctional hydrazide of the present disclosure serves as a one-stop platform that not only enables the enrichment of the EVs but also allows the simultaneous isolation of the EVs and the extraction of EV-derived proteins and EV-derived nucleic acids (NAs) (FIG. 1).

First, the isolation of the EVs may be achieved using a high pH elution buffer. The high pH elution buffer causes the amine groups of the zeolite-NH₂ and the hydrazine groups of the homobifunctional hydrazide to lose positive charge thereof, thereby promoting the isolation of the EVs which were enriched on the zeolite-NH₂ surface. These isolated EVs may be easily filtered through the pores of the syringe filters. As a result, the entire process, including preparation of the zeolite-NH₂-HHs and 10-minute incubation for EV binding, is completed within 15 minutes, enabling the isolation of the highly enriched high-purity EVs.

Second, the extraction of the EV-derived proteins may be achieved using a RIPA lysis buffer, which may lyse EVs. These proteins remain suspended in the RIPA lysis buffer due to the proteins' non-reactivity with the zeolite-NH₂ and homobifunctional hydrazide. As a result, the EV-derived proteins may be simply collected and easily obtained by using the buffer. Third, the extraction of the EV-derived nucleic acids (NAs) may be achieved using a non-chaotropic NP-40 lysis buffer. Chaotropic reagents may cause degradation and potential damage to NAs, so to minimize this, the non-chaotropic NP-40 lysis buffer is used instead of typical RIPA or common lysis buffers. By lysing the EVs with the NP-40 lysis buffer, the EV-derived NAs are released, and the EV-derived NAs may form bonds with the zeolite-NH₂ and added homobifunctional hydrazide. This binding is promoted by electrostatic interactions between the negatively charged EV-derived NAs, the positively charged zeolite-NH₂, and the homobifunctional hydrazide. Additionally, the carbonyl groups of the homobifunctional hydrazide interact with the nucleotide groups (adenine, guanine, and cytosine) and the amino groups of the zeolite-NH₂. Meanwhile, the carbonyl groups of nucleotide groups (guanine, cytosine, thymine, and uracil) react with the amino groups of the zeolite-NH₂ and homobifunctional hydrazide. Additionally, the hydrazide groups of homobifunctional hydrazide react with the carbonyl groups of nucleotide groups (guanine, cytosine, thymine, and uracil). These interactions lead to electrostatic and covalent bonds, effectively trapping EV-derived nucleic acids on the zeolite-NH₂ surface. The bonds between the EV-derived nucleic acids and zeolite-NH₂ and between the EV-derived nucleic acids and homobifunctional hydrazide are broken by the high pH elution buffer, facilitating the extraction of the EV-derived nucleic acids.

Therefore, in yet another aspect, the present disclosure provides a method of isolating extracellular vesicles (EVs) from biological samples by using a composition including a homobifunctional hydrazide and a solid support functionalized with the amine groups.

In yet another aspect, the present disclosure provides a method of isolating extracellular vesicles (EVs) from biological samples, the method including:
(a) adding a composition to biological samples containing extracellular vesicles (EVs);
(b) transferring the resulting mixture obtained through the (a) adding into a syringe and connecting a syringe filter to the syringe;
(c) pressing the syringe to remove unreacted materials and enriching the extracellular vesicles (EVs) bound to zeolite-NH₂ on the surface of the syringe filter;
(d) isolating the extracellular vesicles (EVs) from the zeolite-NH₂ by injecting a high pH elution buffer; and
(e) recovering the isolated extracellular vesicles (EVs). In yet another embodiment, the method may further include incubating the mixture for EV capture after the (a) adding. In yet another embodiment, the method may further include washing the extracellular vesicles (EVs) enriched on the surface of the syringe filter by injecting phosphate-buffered saline (PBS) after the (c) pressing.

In yet another embodiment, in the (b) transferring, the syringe filter may have a pore size of 0.22 to 3 μm. More specifically, the syringe filter may have a pore size of 0.22 to 0.45 μm.

In yet another embodiment, in step (d) isolating, when a RIPA lysis buffer is injected instead of the high pH elution buffer, EV-derived proteins may be extracted. When the RIPA lysis buffer is injected, EVs are lysed, and proteins are released and present in suspension in the RIPA lysis buffer. In this case, the EV-derived proteins may be obtained by recovering the proteins.

In yet another embodiment, in step (d) isolating, when (i) a non-chaotropic lysis buffer is injected instead of the pH elution buffer, and then (ii) a high pH elution buffer is injected, the EV-derived nucleic acids may be extracted. When the non-chaotropic lysis buffer is injected, all components except nucleic acids are released from the EVs, and the nucleic acids remain captured on the zeolite-NH₂ surface. Subsequently, when the high pH elution buffer is injected, the nucleic acids captured on the zeolite-NH₂ surface are released. In this case, the EV-derived nucleic acids may be obtained by recovering the nucleic acids.

In yet another embodiment, the non-chaotropic lysis buffer may be an NP-40 lysis buffer.

In this way, the combination of the solid support functionalized with amine groups and the homobifunctional hydrazide of the present disclosure serves as an efficient tool for the EV isolation and extraction of the EV-derived proteins and nucleic acids which are useful for diagnosis and prognostic assessment of cancer by using clinical specimens.

The present disclosure relates to an extracellular vehicle-isolating composition including a homobifunctional hydrazide and a solid support functionalized with amine groups, and to a method of isolating extracellular vesicles by using the composition. The method may isolate highly enriched high-purity EVs from biological samples and also enable the simultaneous operation for EVs isolation and extraction of EV-derived proteins or EV-derived nucleic acids. The method is low-cost and simple, does not require special equipment other than syringe filters, and allows for high-throughput experiments due to the use of large-volume samples. The method may reduce the time required for protein and nucleic acid extraction by simplifying the experiments. Additionally, this EV enrichment may increase the sensitivity of biomarker detection for disease diagnosis and treatment.

Hereinafter, the configuration and effects of the present disclosure will be explained in more detail through examples. However, these examples are only for illustrative purposes of the present disclosure, and the scope of the present disclosure is not limited by these examples.

### EXAMPLES

### Experiment Materials

The following experimental materials were commonly applied to each example described below.

Zeolite (#96096), 3-aminopropyl (diethoxy) methylsilane (APDMS, #371890), glutaraldehyde (#340855), and sodium bicarbonate (#S5761) were purchased from Sigma-Aldrich.

Malonic acid dihydrazide (#M3206) was purchased from Tokyo

Chemical Industry. Adipic acid dihydrazide (#A0638) was purchased from Sigma-Aldrich. Oxalyl dihydrazide (#131296) was purchased from Sigma-Aldrich. Succinic acid dihydrazide (#S5502) was purchased from Sigma-Aldrich. Carbonic dihydrazide (#C11006) was purchased from Sigma-Aldrich.

Kovax 1 to 30 ml syringes and BD 1 ml syringes (#309628) were purchased from Korea Vaccine and Becton, Dickinson and Company, respectively.

Polytetrafluoroethylene (PTFE) 3 μm syringe filters (#18140), PTFE 1 μm syringe filters (#16278), polyvinylidene fluoride (PVDF) 3 μm syringe filters (#18215), and PVDF 1 μm syringe filters (#18214) were purchased from Tisch Scientific. PVDF 0.22 μm syringe filters (#FJ25ASCCA002DL01) and PVDF 0.45 μm syringe filters (#FJ25ASCCA004FL01) were supplied by GVS Filter Technology. 100X antibiotic-antifungal cocktail (#15240062), exosome-depleted fetal bovine serum (FBS, #A2720803), and Dulbecco's modified Eagle medium (DMEM, #41965039) were purchased from Gibco.

Total exosome isolation reagents (#4478359) and a SuperScript IV First-Strand Synthesis System (#18091050) were supplied by Invitrogen.

Antibodies included rabbit anti-CD9 antibodies (#ab236630), rabbit anti-CD63 antibodies (#ab134045), mouse anti-CD81 antibodies (#ab79559), rabbit anti-ARF6 antibodies (#ab131261), rabbit anti-GRP78 antibodies (#ab108615), rabbit anti-GM130 antibodies (#ab52649), goat anti-rabbit IgG/HRP antibodies (#ab205718), goat anti-mouse IgG/HRP antibodies (#ab6789), and donkey anti-rabbit IgG/Gold antibodies (#ab39597), all the antibodies being from Abcam Plc, and also included rabbit anti-Calnexin antibodies (#2679S) from Cell Signaling Technology.

A RIPA lysis and extraction buffer (#89901) from Thermo Scientific and an NP-40 lysis buffer (#J60766) from Alfa Aesar were used to lyse EVs.

A Mir-X miRNA qRT-PCR TB Green Kit (#638314) was purchased from Takara. A Brilliant III SYBR Green QPCR Master Mix (#600882) was supplied by Agilent Technologies. Oligonucleotides used were purchased from BIONICS and Macrogen.

### Example 1

### Synthesis of Zeolite-NH₂

Zeolite-NH₂ was synthesized in accordance with a slightly modified protocol from a previously reported method. A total of 3 g of zeolite was washed twice for 10 minutes at 550 rpm by using distilled water and 95% ethanol. To ensure uniformity of zeolite size, larger-sized particles that precipitated within 1 minute were removed in the first washing. After each washing, the washed zeolite was briefly centrifuged at 1,000 rpm for 10 seconds, and the supernatant was discarded to remove smaller-sized particles. Amine group functionalization was then performed by incubating the zeolite in a 2% APDMS solution in 95% ethanol for 4 hours at 450 rpm. The synthesized zeolite-NH₂ was washed three times for 5 minutes at 550 rpm using distilled water and 95% ethanol. After these, the zeolite-NH, was left to dry completely in a vacuum chamber for at least 24 hours. The dried zeolite-NH₂ was stored at room temperature until further use.

### Example 2

### Biological Sample Preparation

Human colon cancer cell line HCT116 (ATCC #CCL-247) was purchased from American Type Culture Collection. The cells were cultured under standard conditions of a temperature of 37° C. and 5% CO₂. A DMEM medium was supplemented with 10% exosome-depleted FBS and an antibiotic-antifungal cocktail. The cells were grown to isolate EVs from the HCT-116 cell culture medium until the cells reached approximately 80% confluence. The medium was centrifuged at 400 g for 30 minutes at a temperature of 4° C. to obtain cell-free supernatant. After filtering the cell-free supernatant by using a PVDF 0.22 μm syringe filter, the filtered cell-free supernatant was immediately used to isolate EVs by using a zeolite-NH-homobifunctional hydrazide (HH) combination described below or to isolate EVs by an ultracentrifugation (UC) method and a total exosome isolation (TEI) method. Alternatively, the filtered cell-free supernatant was stored at a temperature of −20° C. for up to 4 weeks.

### Example 3

### Extracellular Vesicle (EV) Isolation by Using Zeolite-NH₂-HH Combination, and Extraction of EV-Derived Proteins or EV-Derived Nucleic Acids

The workflow of combining zeolite-NH₂ and HH is schematically shown in FIG. 1. First, 5 mg/ml of zeolite-NH₂ and 25 mg/ml of homobifunctional hydrazide were mixed per 1 ml of the prepared biological sample, and the mixture was incubated for 10 minutes. This mixture was then transferred to a Kovax syringe of appropriate volume, and the syringe with the mixture was connected to a PVDF 0.45 μm syringe filter. All unreacted waste was removed by gently pressing the syringe by hand. Then, the enriched EVs bound to zeolite-NH₂ on the surface of the syringe filter were washed with 3 ml of PBS. Residual PBS in the syringe filter was removed with air. EV isolation was performed by injecting 300 μl of high pH elution buffer containing 10 mM sodium bicarbonate at a pH of 10.4 by using a BD 1 ml syringe and incubating the sample with the high pH elution buffer for 1 minute. Afterward, the EVs isolated from the zeolite-NH₂ surface were isolated by injecting air.

For EV-derived protein extraction, 300 μl of RIPA lysis buffer was injected by using a BD 1 ml syringe, and the sample with the RIPA lysis buffer was incubated at a temperature of 4° C. for 20 minutes. The EV-derived proteins in RIPA lysis buffer were then extracted by injecting air.

For EV-derived nucleic acid (NA) extraction, 300 μl of NP-40 lysis buffer containing 7.5 mg of MDH was injected by using a BD 1 ml syringe, and the sample with the NP-40 lysis buffer was incubated for 20 minutes at room temperature. After removing the EV-derived proteins through air injection, the residual EV-derived proteins in the sample were washed with 3 ml of PBS, and then the residual PBS in the filter was removed. Lastly, the EV-derived NAs were eluted by injecting 300 μl of high pH elution buffer by using a BD 1 ml syringe and incubating the EV-derived nucleic acids for 1 minute. The NAs isolated from the zeolite-NH₂ surface were isolated by injecting air.

### Example 4

### Characterization of Zeolite-NH₂-HH Combination

The properties of the zeolite-NH₂-HH (herein, homobifunctional hydrazide (HH) is assumed to be MDH) combination were confirmed through SEM, DLS, FT-IR, and zeta potential analysis. FIGS. 2A and 2B show SEM images of synthesized zeolite-NH₂ and EVs captured on the zeolite-NH₂. Zeolite maintained structural stability and achieved uniformity during zeolite-NH₂ synthesis. In addition, it was confirmed that in the presence of MDH, EVs with a size of 121.54 to 184.81 nm were successfully captured on the zeolite-NH₂ surface.

The zeolite-NH₂ had a uniform size distribution with an average of 3,833.4±529.7 nm (FIG. 2C). The FT-IR spectra of the zeolite-NH₂, zeolite-NH₂-MDH, zeolite-NH₂-MDH-EV, and pure MDH are shown in FIG. 2D. As shown in FIG. 2D, the FT-IR analysis of the zeolite-NH₂ showed that zeolite-specific peaks were made at 461 cm⁻¹ (symmetric bending of tetrahedral bonded Si or Al), 550 cm⁻¹ (symmetric stretching of a double six-membered ring), 665 cm⁻¹ (symmetric stretching of Si—O—Si), and a broad 974 cm⁻¹ (asymmetric stretching of Si—O—Al and Si—O—Si). Additional peaks at 1665 cm⁻¹ (NH bending) and broad 3462 cm⁻¹ (NH stretching) were made due to functionalization with amine groups. Additionally, in the zeolite-NH₂-MDH combination method, there was the presence of specific additional peaks at 1418 cm⁻¹ (NH bending and CN stretching) and 1533 cm⁻¹ (CN stretching) and increased peaks at 1665 cm⁻¹ (NH bending) and a broad 3462 cm⁻¹ (NH stretching). The presence of the displayed peaks indicated that the MDH reacted with the zeolite-NH₂, exerting the MDH's specific properties on the zeolite surface. The enhanced intensity of the peak at 1665 cm⁻¹ indicated that imine bonds were formed through the reaction of zeolite-NH₂ and MDH.

Additionally, the zeta potential values of the zeolite-NH₂, zeolite-NH₂-MDH, and zeolite-NH₂-MDH-EV are shown in FIG. 2E. As shown in FIG. 2E, the surface charge of the zeolite-NH₂ at +7.51 mV increased to a higher value (+9.99 mV) due to the hydrazine group (NH-NH₂) Of MDH. This suggested that the zeolite-NH₂-MDH combination would have a more robust electrostatic interaction and binding potential with negatively charged EVs. When the zeolite-NH₂-MDH combination was bound to the EVs, the peaks representing both the amine groups and the MDH's functional groups decreased. This meant that the combination of the negatively charged EVs and the positively charged zeolite-NH₂-MDH reduced the strength of a reactor. Due to the binding of the zeolite-NH₂-MDH combination with the negatively charged EVs, the surface charge of the zeolite-NH₂-MDH-EV combination was reduced to −17.47 mV. These results showed that the zeolite-NH₂-MDH combination could serve to successfully capture EVs.

### Example 5

### Efficiency Comparison Among Different Types of Homobifunctional Hydrazide

To determine and compare the efficiencies of different types of homobifunctional hydrazide, extraction of EV-derived proteins and EV-derived nucleic acids using different types of homobifunctional hydrazide was performed with a cell-free supernatant derived from HCT116 cells. First, the concentration of the EV-derived proteins was measured, the EV-derived proteins having been extracted by using adipic acid dihydrazide, carbonic dihydrazide, malonic dihydrazide, oxalyl dihydrazide, and succinic acid dihydrazide, respectively.

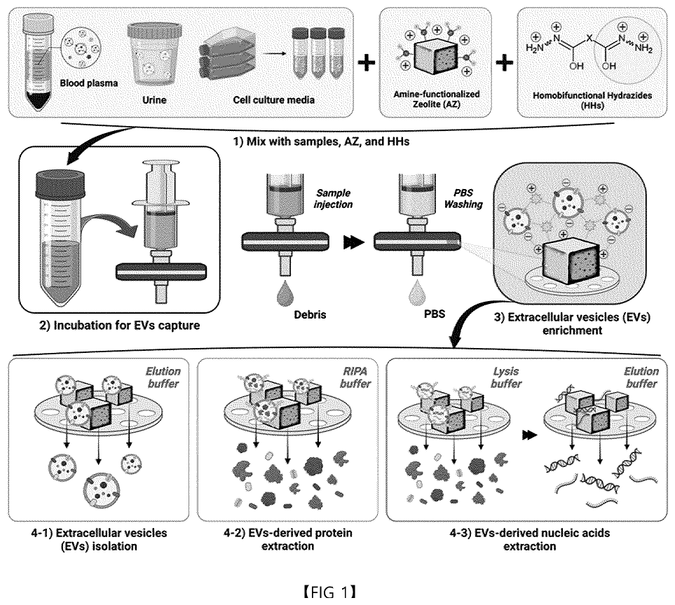

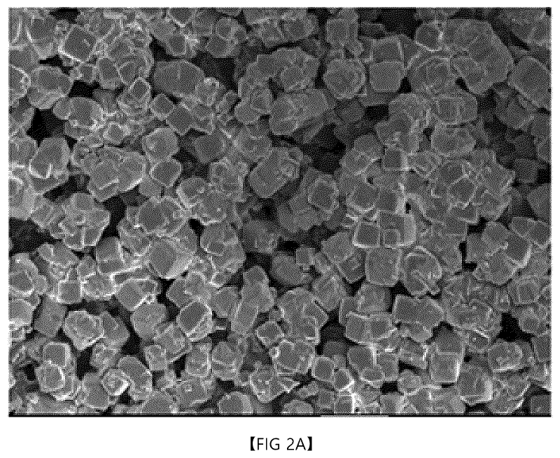

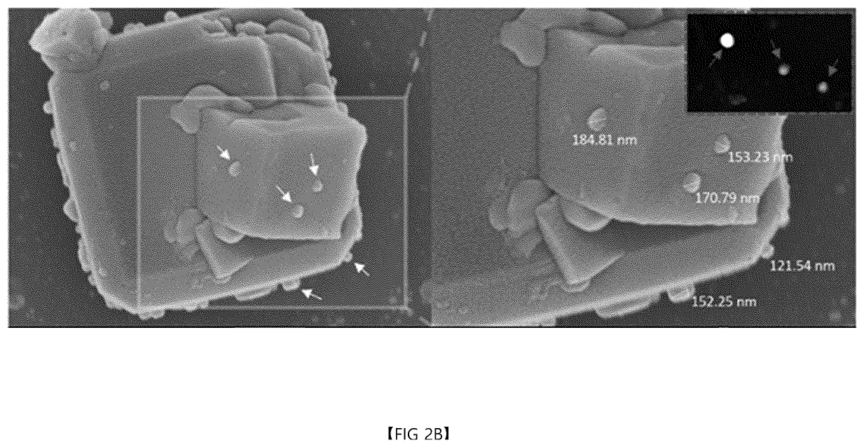

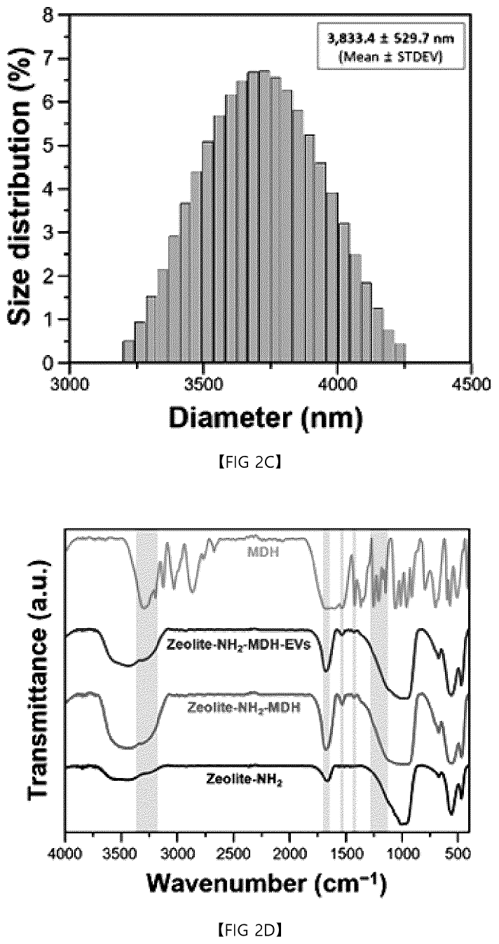

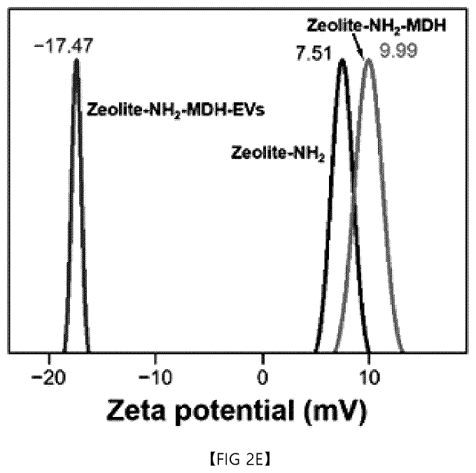

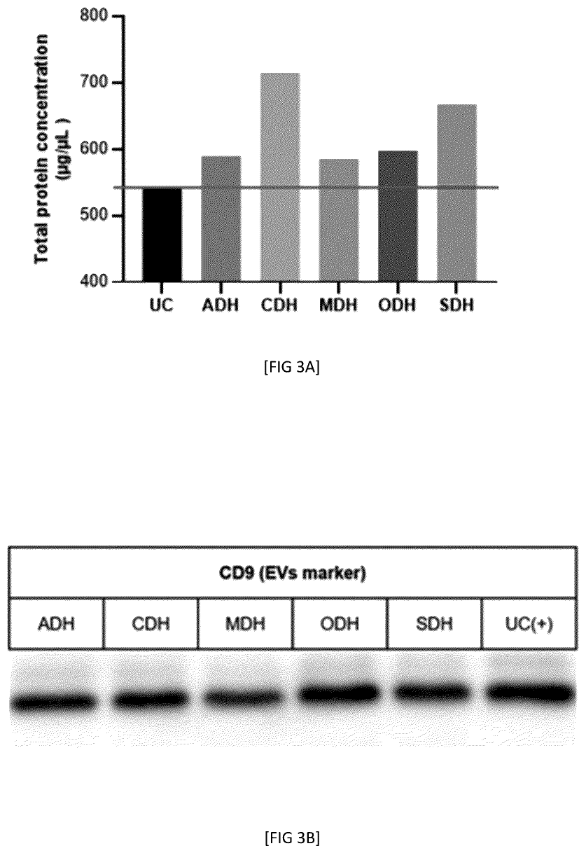

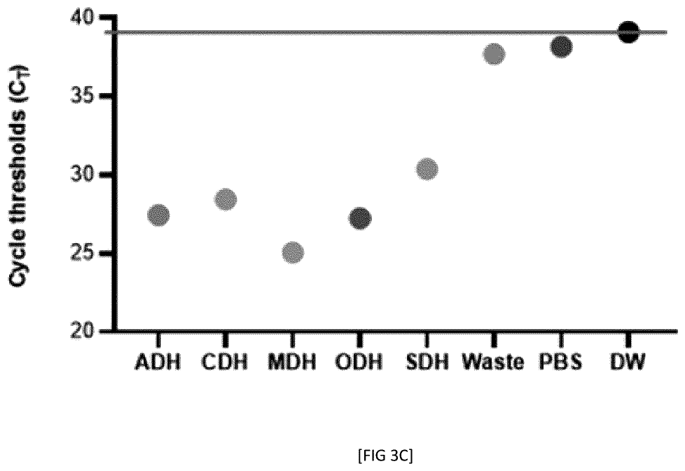

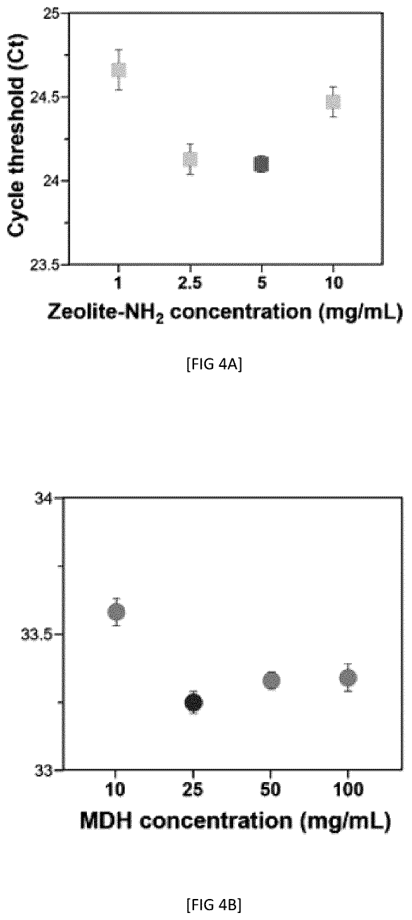

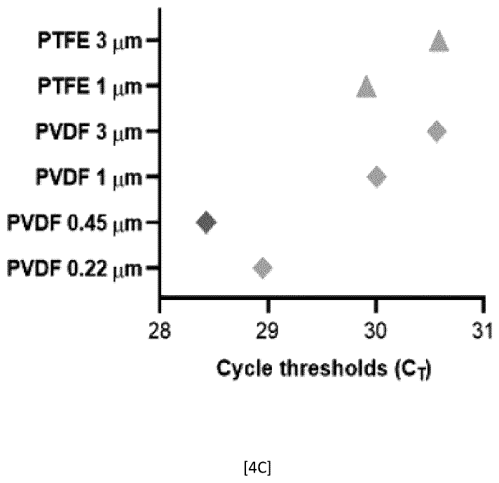

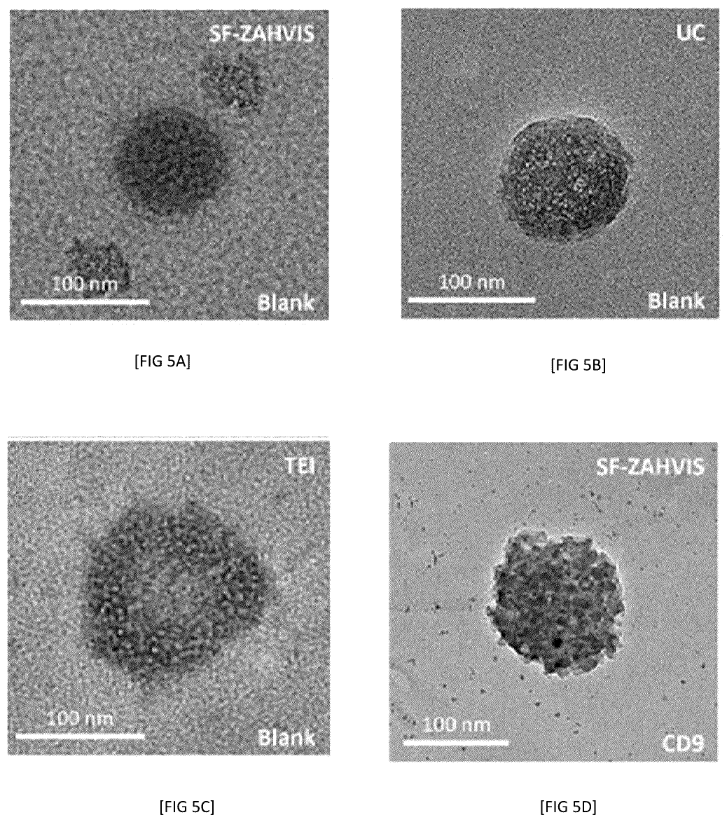

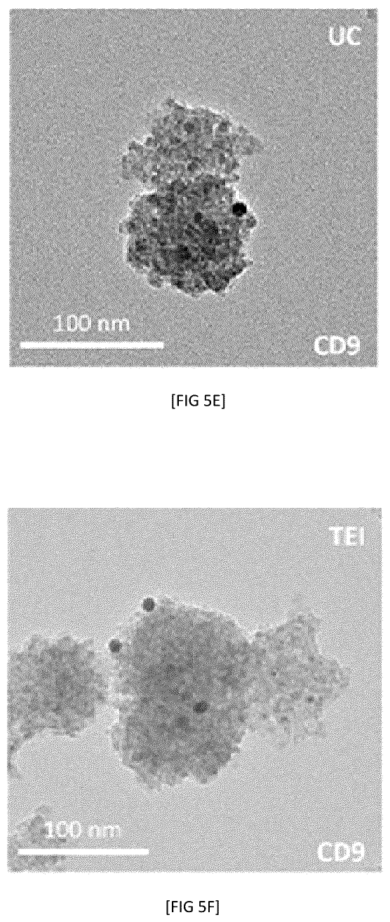

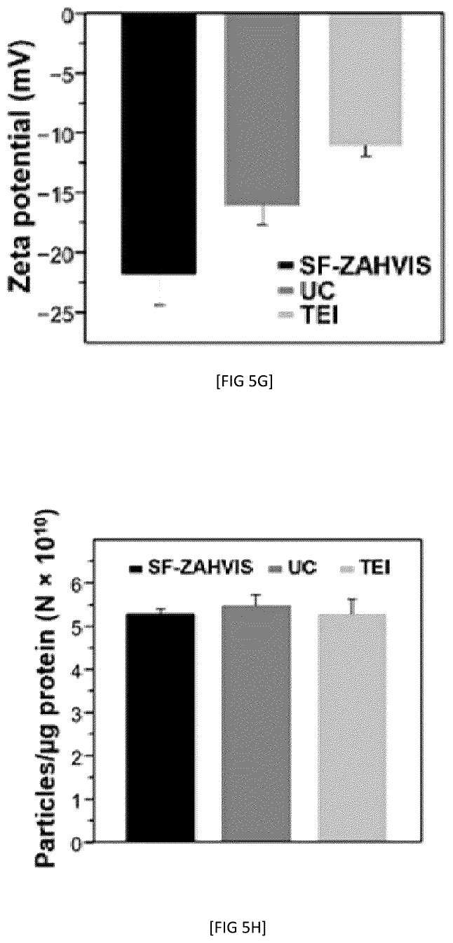

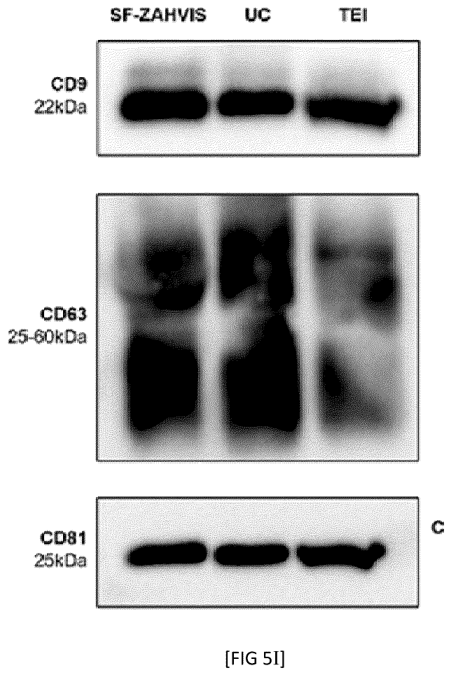

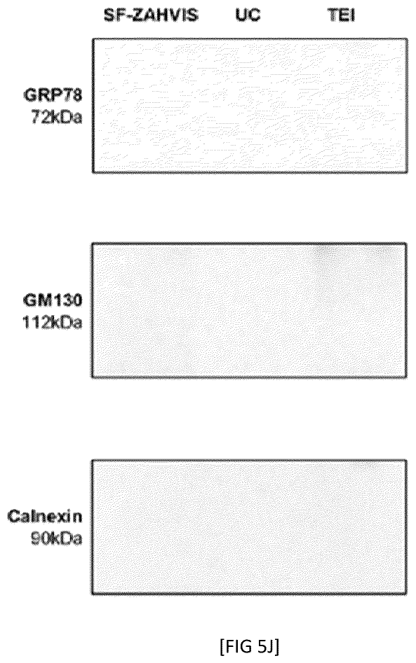

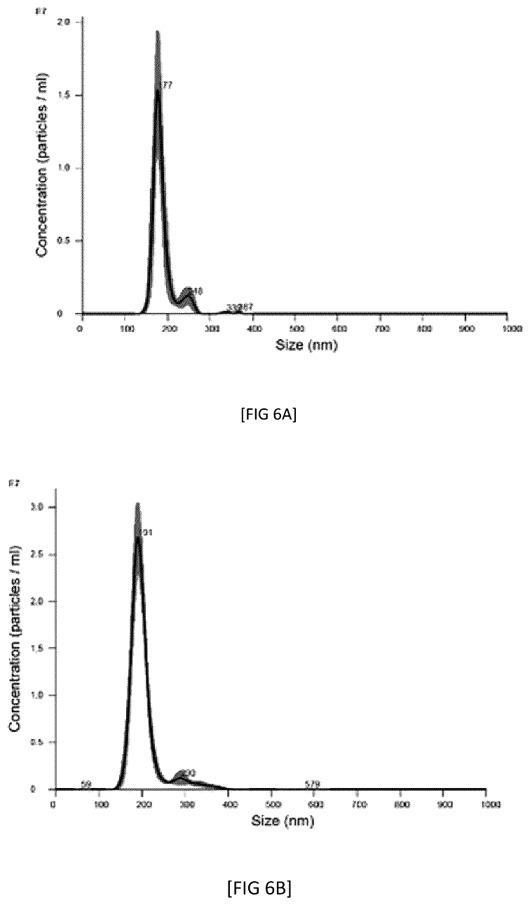

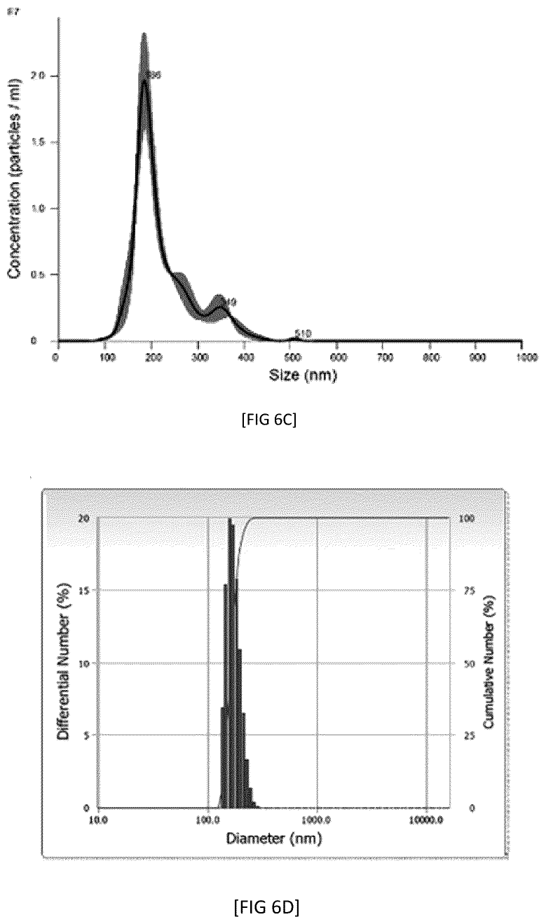

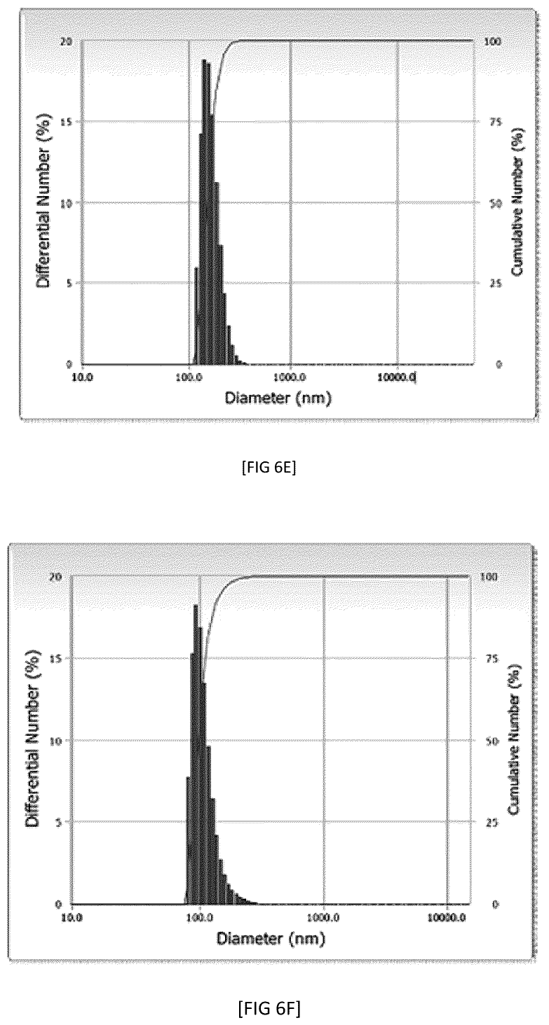

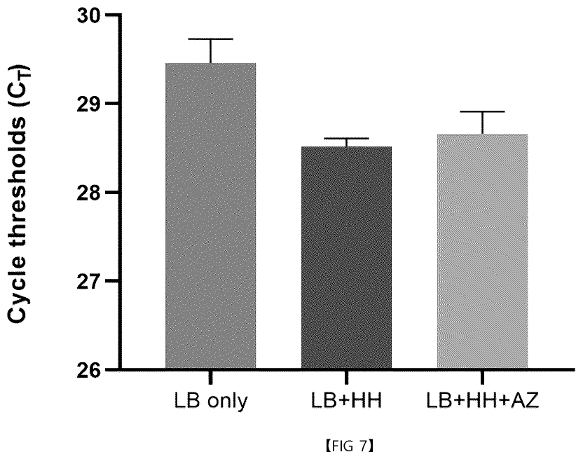

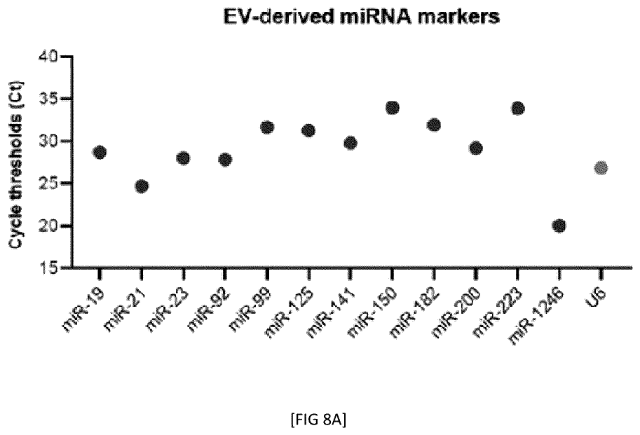

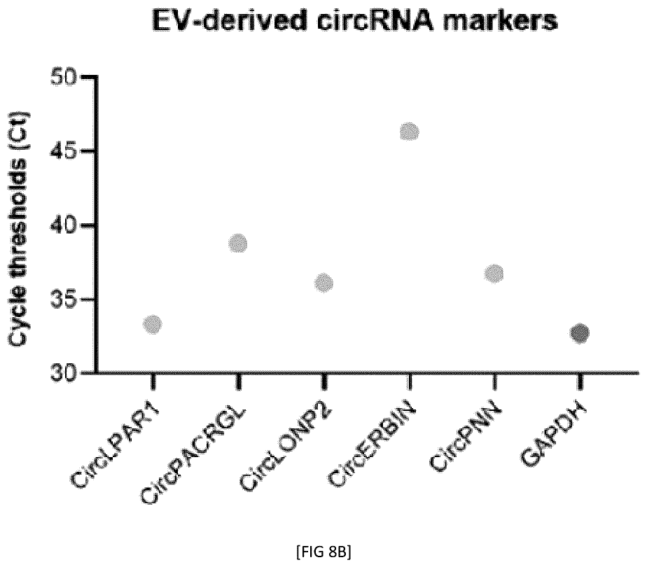

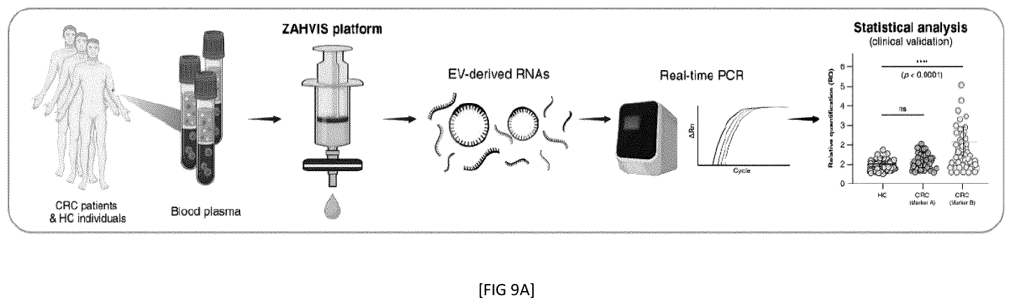

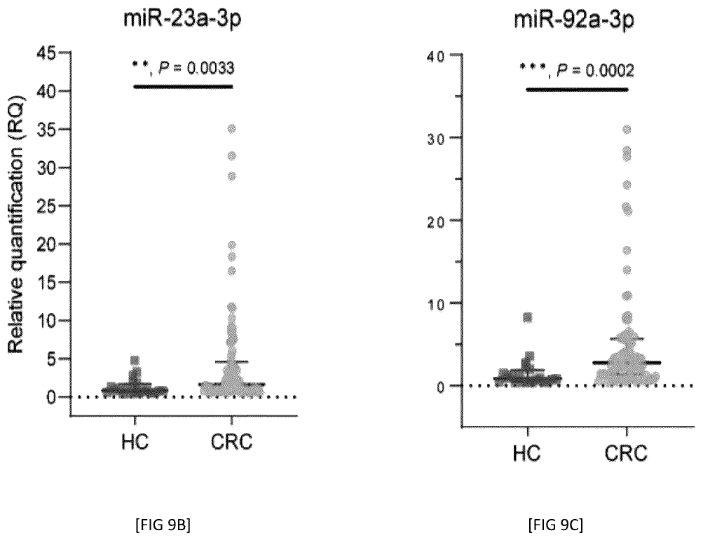

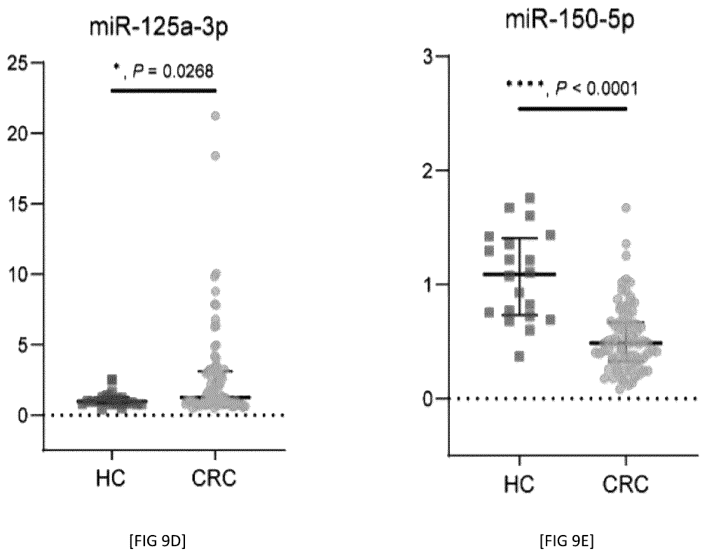

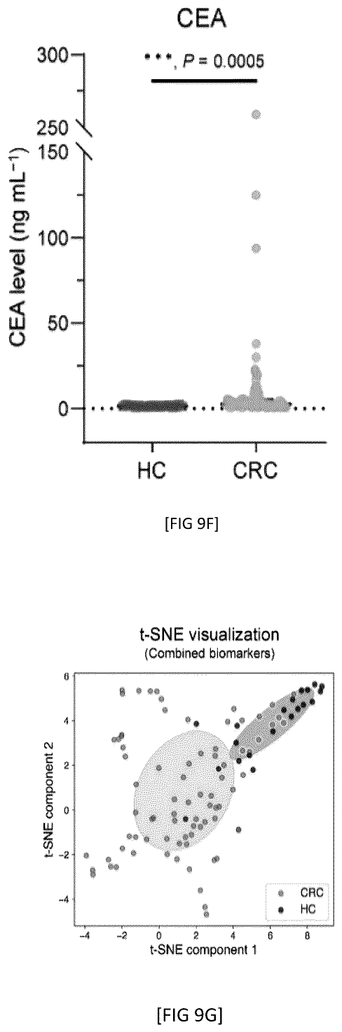

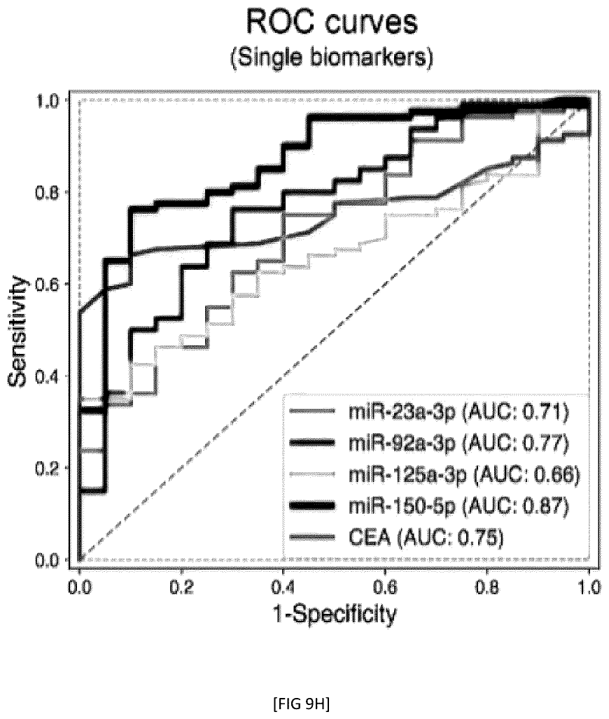

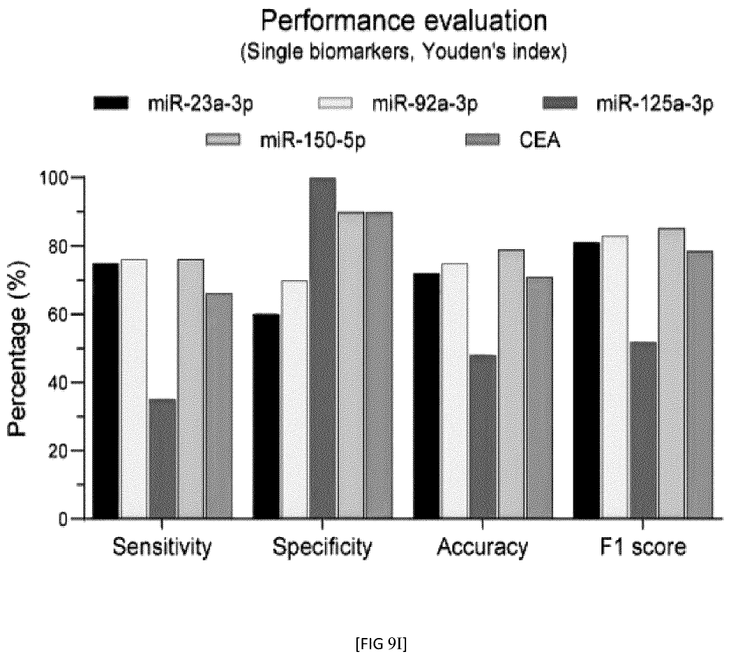

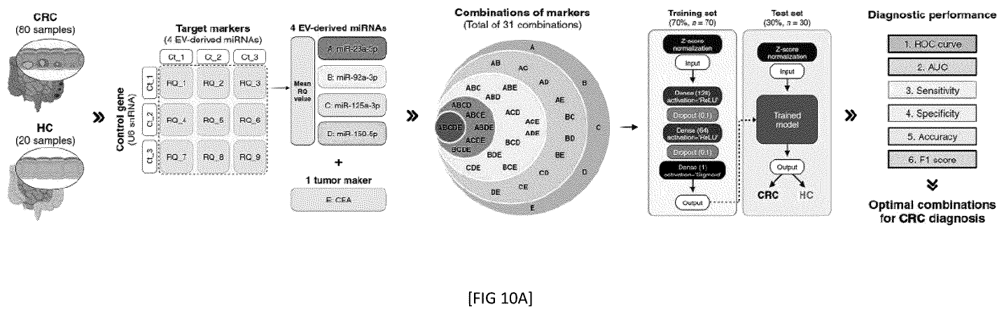

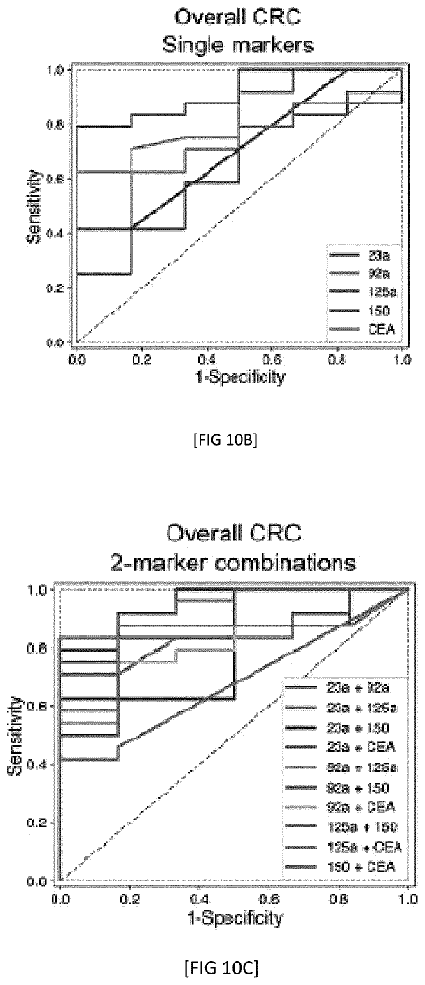

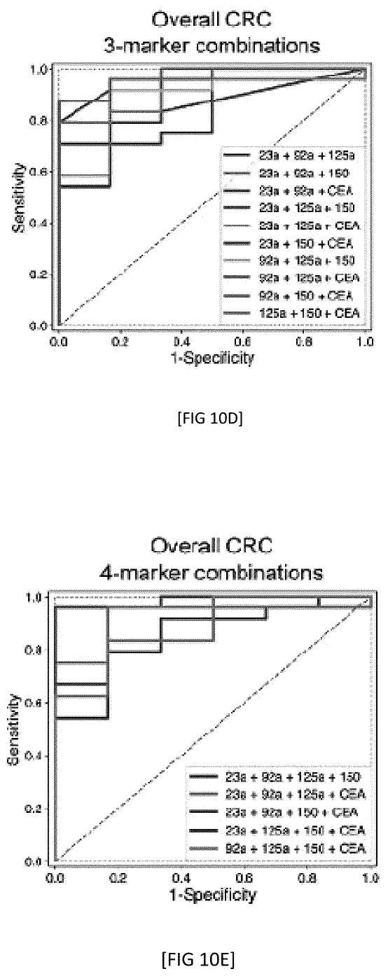

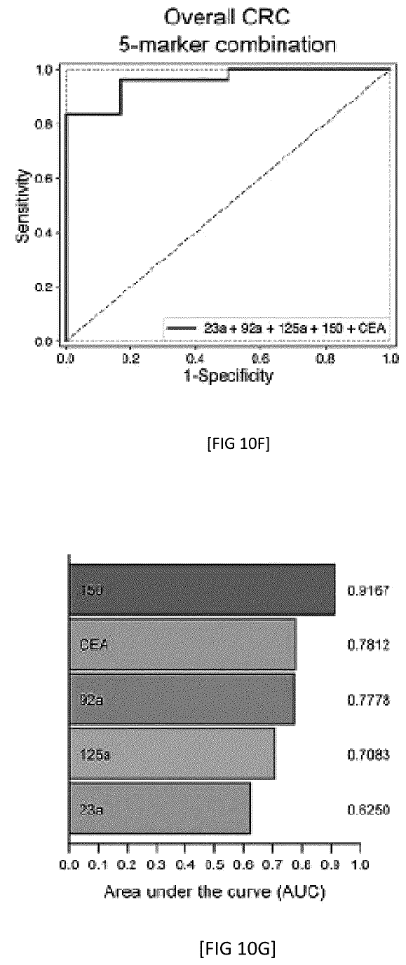

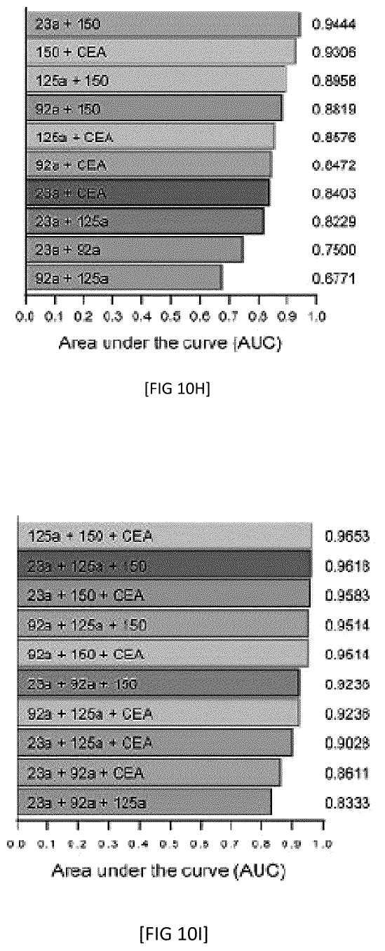

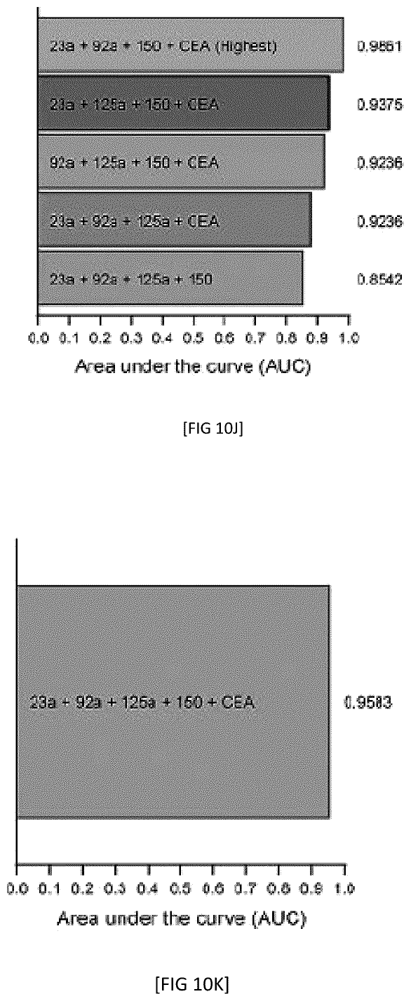

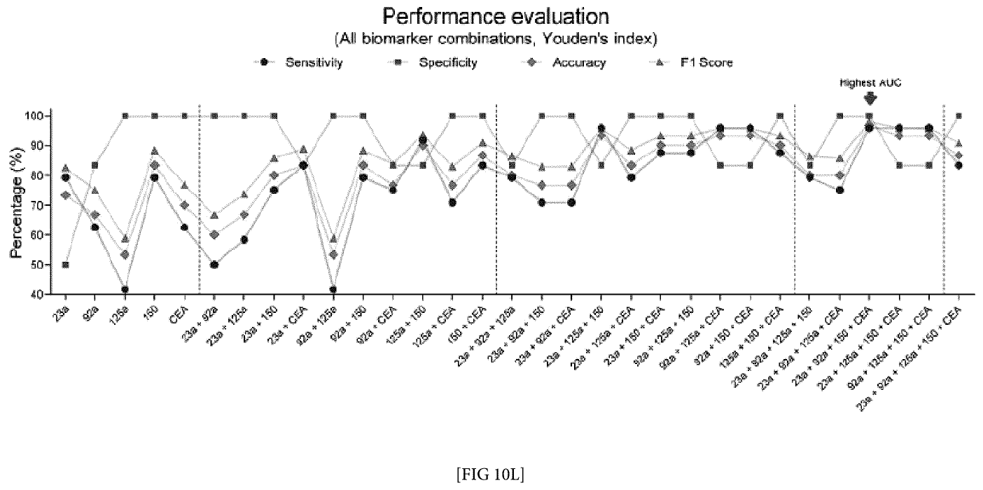

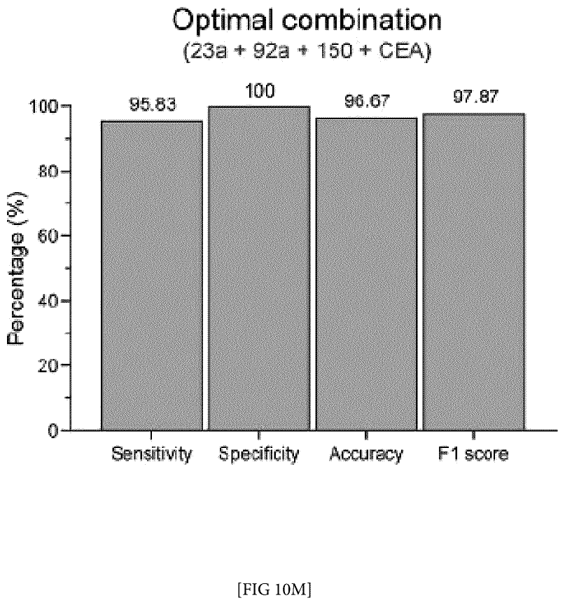

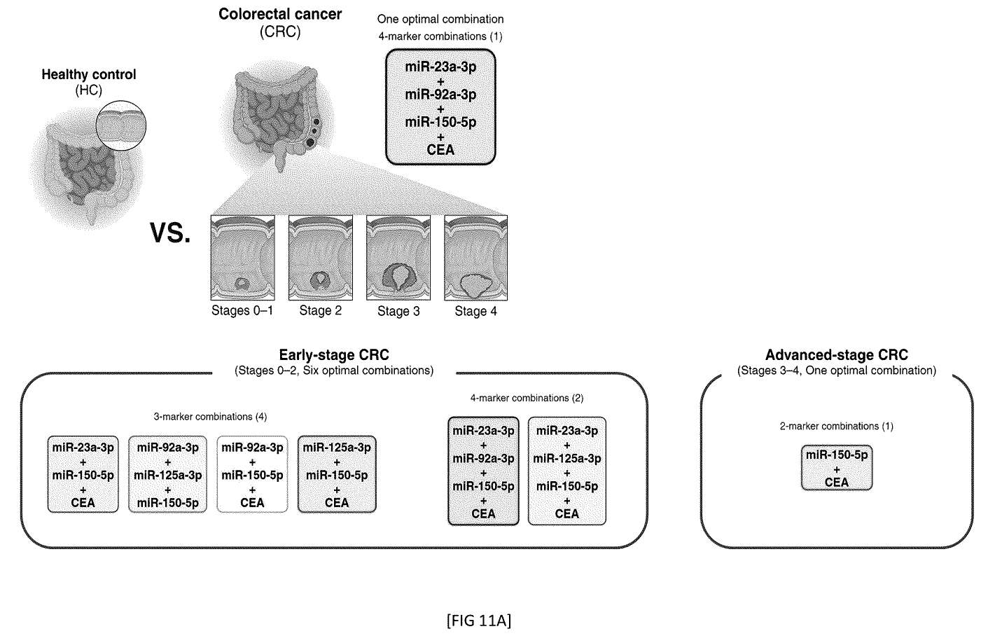

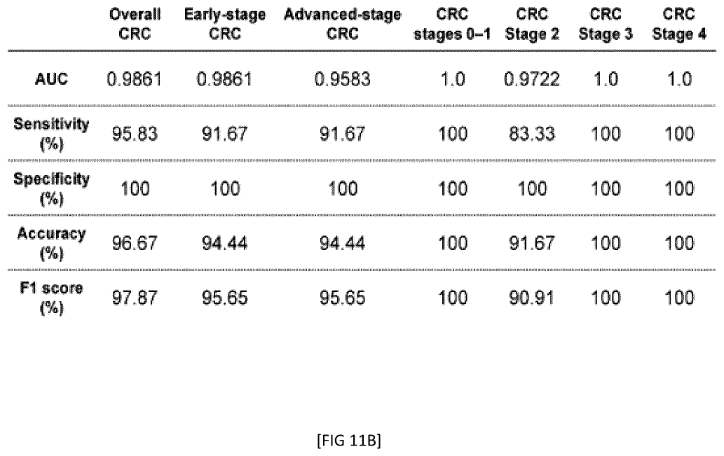

As a result, the concentration of the EV-derived proteins was found to be high in that order of use of carbonic dihydrazide, succinic acid dihydrazide, oxalyl dihydrazide, adipic acid dihydrazide, and malonic acid dihydrazide, and a larger amount of the EV-derived proteins could be extracted than the conventional ultracentrifugation (UC) method (FIG. 3A). In addition, the expression of CD9, which was an EV-specific surface protein, was confirmed during EV-derived protein extraction with all candidate substances, thereby the expression level of the EV-derived proteins was confirmed to be similar to that obtained in the UC method (FIG. 3B). Next, the efficiency of EV-derived nucleic acid extraction was confirmed by detecting miR-21 in EV-derived nucleic acids which were extracted with all candidate substances by real-time PCR (qPCR). As a result, the extraction efficiency of the EV-derived nucleic acids was found to be high in the order of use of malonic acid dihydrazide, oxalyl dihydrazide, adipic acid dihydrazide, carbonic dihydrazide, and succinic acid dihydrazide (FIG. 3C). In addition, the qPCR results of the EV-derived nucleic acids lost in PBS for washing after the use of NP-40 lysis buffer for EV lysis were similar to those obtained in the case of distilled water (DW). Thus, it was confirmed that the zeolite-NH₂-HH combination maintained high binding affinity to the EV-derived nucleic acids, resulting in almost no loss of the EV-derived nucleic acids (see Waste, PBS, and DW in FIG. 3C). These results showed that it was possible to extract EV-derived proteins and EV-derived nucleic acids with high efficiency with all candidate substances.

### Example 6

### Optimization of Zeolite-NH₂-MDH Combination

To determine the optimal conditions for efficient EV isolation using the zeolite-NH₂-MDH combination, several key were optimized, including the concentrations of parameters zeolite-NH₂ and MDH, and the type and pore size of the syringe filter.

The present inventors found that the isolation efficiency increased as the concentration of zeolite-NH₂ was increased from 1 to 5 mg/ml, but the isolation efficiency decreased when the concentration was further increased to 10 mg/ml (FIG. 4A). This suggested that increasing the concentration of zeolite-NH₂ to 5 mg/ml increased the surface area available for EV binding. Meanwhile, the result also suggested that excessive concentration of zeolite-NH₂ might reduce the contact opportunities between zeolite-NH₂ and EVs due to insufficient mixing.

The concentration of homobifunctional hydrazide (herein, homobifunctional hydrazide (HH) is assumed to be MDH) was an important factor affecting the increase in bond strength between the zeolite-NH₂ and EVs. The hydrazide group of the homobifunctional hydrazide served d as a positively charged functional group with the ability to form a variety of covalent bonds while also acting as a potential competitor to the amine group. Therefore, it was essential to use an appropriate concentration of homobifunctional hydrazide. As shown in FIG. 4B, the isolation efficiency peaked at an MDH concentration of 25 mg/ml but slightly decreased as the concentration further increased.

Meanwhile, the isolation efficiency was further evaluated depending on the type and pore size of the syringe filter used for the zeolite-NH₂-HH combination. Both PTFE and PVDF syringe filters were chemically stable, exhibited high durability, and were widely used in biological applications. FIG. 4C shows that the PVDF 0.45 μm syringe filter showed the highest isolation efficiency. These results indicated that the EV isolation efficiency was more affected by the pore size than the type of the syringe filter and that the pore size was smaller, the isolation efficiency was generally higher. The decrease in isolation efficiency was observed when the filters had larger-sized pores. This might be due to the loss of EVs, which might occur when the zeolite-NH₂ that captured EVs during the experiments passed through the larger-sized pores of the filters. Therefore, the optimal concentrations of zeolite-NH₂ and MDH were determined to be 5 mg/ml and 25 mg/ml, respectively, and the optimal filter was found to be a PVDF 0.45 μm syringe filter. These optimized conditions were applied to all subsequent experiments.

### Example 7

### Comparative Evaluation of Zeolite-NH₂-HH Combination Method with Conventional Methods

The efficacy of the zeolite-NH₂-HH combination method was evaluated in comparison with those obtained by commonly used EV isolation techniques, especially ultracentrifugation (UC) and total exosome isolation (TEI) methods.

To perform experiments by the zeolite-NH₂-HH combination method, the method described in Example 3 was used as it is. In the experiments by the UC method, 10 ml of cell culture medium was centrifuged at 110,000 g for 70 minutes at a temperature of 4° C., and the supernatant was discarded, leaving behind an EV pellet. In the experiments by the TEI method, 10 ml of cell culture medium was mixed with 5 ml of TEI reagent, the mixture was then incubated overnight at a temperature of 4° C., centrifuged at 10,000 g for 60 minutes at a temperature of 4° C., and the supernatant was discarded. In both UC and TEI methods, the EV pellets were reconstituted with 300 μl of PBS for EV collection, or the EV pellets were reconstituted with 300 μl of RIPA lysis buffer for EV-derived protein extraction.

The morphology of the EVs isolated by each method (zeolite-NH₂-HH combination method, UC method, and TEI method) were evaluated through TEM and SEM images. For TEM imaging, the EVs were diluted 1:10 in PBS and incubated on Formvar/carbon-coated copper grids for 30 minutes at a temperature of 37° C. For CD9 labeling, grids were initially blocked with 5% BSA for 20 minutes, followed by incubation overnight at a temperature of 4° C. with 10 nm gold-tagged CD9 antibodies which were diluted in accordance with the manufacturer's instructions. For both blank and CD9 labeled images, grids were treated with 2.5% glutaraldehyde and 3% citrate solutions for 5 minutes each, washed with distilled water (DW), and placed on Parafilm. The grids were left to dry overnight in a fume hood. TEM images were visualized with a JEM-ARM200F device (JEOL, Japan). For SEM imaging, the EVs were diluted at a ratio of 1:10 in PBS and dropped onto silicon wafers for 30 minutes at a temperature of 37° C. The silicon wafers were then fixed using 2.5% glutaraldehyde for 10 minutes. The silicon wafers were soaked in various percentages of ethanol (30%, 50%, 70%, 80%, 90%, and 100%) for 15 minutes each time. After drying the silicon wafers in the fume hood, the silicon wafers were covered with a thin layer of platinum (Pt), and images were captured using a JSM-7610F-Plus device (JEOL).

The morphology of the EVs isolated by each method was confirmed through blank TEM and SEM images. All cases showed spherical EVs approximately 100 nm in size (FIGS. 5A to 5C). In addition, the presence of CD9, which was a specific marker protein of the tetraspanin family of EVs, was confirmed by using 10 nm gold particles for immunolabeling (FIGS. 5D to 5F).

Next, NTA, DLS, and zeta potential analyzes were performed in accordance with standard protocols to determine the number, diameter distribution, and surface properties of the EVs. The EVs isolated by each method were resuspended in PBS at a ratio of 1:50 or 1:100 and injected into a cuvette. In the NTA analysis, the concentration and intensity distribution of the EVs were measured by using NS300 instrument (Malvern Panalytical) and NanoSight software (Malvern Panalytical). In the DLS analysis, the number distribution of the EVs was measured by using ELS-z1000 instrument (Otsuka Electronics, Japan) and Photal software (Otsuka Electronics). The surface charge of the EVs was measured with a NANO ZS 90 instrument (Malvern Panalytical).

As shown in FIGS. 6A to 6C, when the particle size distribution of the EVs was determined based on signal intensity in the NTA analysis, the particle size distribution of the EVs was found to be 187.1±27.5 nm in the zeolite-NH₂-HH combination method, 202.4±36.7 nm in the UC method, and 222.5±65.3 nm in the TEI method. In addition, as shown in FIGS. 6D to 6F, when the particle size distribution of the EVs was determined based on particle number in the DLS analysis, the particle size distribution of the EVs was found to be 172.2±34.0 nm in the zeolite-NH₂-HH combination method, 176.3±26.2 nm in the UC method, and 114.2±26.7 nm in the UC method.

Additionally, as shown in FIG. 5G, the isolated EVs had a zeta potential in a range of −10 and −25 mV (−21.1±2.57 mV for the zeolite-NH₂-HH combination method, −15.53±1.56 mV for the UC method, −10.67±0.91 mV for the TEI method) and exhibited a negative charge.

The ratio of particle number to protein concentration of the isolated EVs was an important indicator of the concentration and purity of the EVs. A higher ratio suggested successful separation due to increased particle concentration. As shown in FIG. 5H, in the zeolite-NH₂-HH combination method, a particle/protein concentration ratio was shown to be 5.26×10¹⁰±16×10⁹. This suggested that the zeolite-NH₂-HH combination method was comparable to the UC method (5.45×10¹⁰±2.68×10⁹) and the TEI method (5.27×10¹⁰±3.19×10⁹). Therefore, the zeolite-NH₂-HH combination method allowed for highly enriched high-purity EV isolation which was similar to conventional EV isolation techniques.

Next, to determine the quantity and purity of EV-derived proteins extracted by the zeolite-NH₂-HH combination method and confirm the presence of specific EV proteins, western blotting analysis was performed for EV-specific proteins (CD9, CD63, and CD81) as well as non-specific proteins (GRP78, GM130, and

Calnexin).

Specifically, the EV-derived proteins extracted with a RIPA lysis buffer were analyzed in each case of the zeolite-NH₂ and HH combination method, UC method, and TEI method. Protein concentration was determined using a Bradford assay by using serial dilutions of BSA as a standard. Equal amounts (20 μg) of the extracted proteins were placed, the proteins were separated on 10% SDS-PAGE and transferred to PVDF microporous membranes. The membranes were blocked in PBS-Tween 20 containing 5% skim milk for 1 hour. Primary antibodies (CD9, CD63, CD81, ARF6, GRP78, GM130, and Calnexin) were diluted in accordance with the manufacturer's instructions and incubated overnight at a temperature of 4° C. The membranes were then placed in HRP-tagged secondary antibody solution for 1 hour which had been diluted again in accordance with the manufacturer's instructions. Marker proteins were detected using a 1:1 mixture of peroxidase and chemiluminescent substrate. Images were captured with the ChemiDoc MP Imaging System (Bio-Rad) and Image Lab software (Bio-Rad).

The results of the Western blotting analysis are shown in FIG. 5I. As shown in FIG. 5I, the presence of representative extracellular vesicle (EV) markers belonging to the tetraspanin family, namely CD9, CD63, and CD81, was confirmed in all three technique cases of the zeolite-NH₂-HH combination method, UC method, and TEI method. Additionally, the presence of apoptotic body marker GRP78, Golgi marker GM130, and endoplasmic reticulum (ER) marker Calnexin was not observed, which meant that there were no contaminating proteins in the isolated EVs (FIG. 5J).

Therefore, the zeolite-NH₂-HH combination method was demonstrated to be an effective approach for EV isolation and EV-derived protein extraction, thereby the combination method allowed the achievement of purity and concentration comparable to the established UC and TEI methods.

Furthermore, the extraction efficiencies of the EV-derived nucleic acids were evaluated depending on the addition of additional homobifunctional hydrazide (HH) and zeolite-NH₂ (AZ) when the EV-derived nucleic acids were extracted. Specifically, in injecting the NP-40 lysis buffer (LB) used for the EV-derived nucleic acid extraction, these three cases were designed: injection of only LB (LB only), injection of homobifunctional hydrazide mixed LB with (LB+HH), and injection of homobifunctional hydrazide and zeolite-NH₂ (AZ) mixed with LB (LB+HH+AZ). Afterward, the EV-derived nucleic acid extraction efficiencies among the three cases were compared. As a result, as shown in FIG. 7, it was confirmed that when HH was added to LB, the EV-derived nucleic acids could be extracted more efficiently than when only LB was used. This showed that the added HH provided an additional reactive group and increased the binding force of AZ to nucleic acids which were released from the EVs. However, regarding AZ, it was confirmed that the extraction efficiencies of EV-derived nucleic acids did not increase when additional AZ was added. This showed that the AZ amount used for enriching the EVs was sufficient so that there was no need for additional solid supports to capture the EV-derived nucleic acids.

### Example 8

### Detection of EV-Derived Nucleic acid Biomarkers for colorectal cancer (CRC) using zeolite-NH₂-HH combination

To evaluate the capacity of zeolite-NH₂-HH combination in EV-derived nucleic acid (NA) extraction and detect colorectal cancer (CRC)-related biomarkers, non-coding RNAs from EVs extracted from the cell culture medium of the CRC cell line HCT116 were analyzed using real-time PCR (qPCR).

A Mir-X miRNA qRT-PCR TB Green Kit was used for miRNA cDNA synthesis and qPCR analysis. 4 μl of EV-derived NAs obtained from the samples using a zeolite-NH₂-HH combination were mixed with 5 μl of reaction buffer and 1 μl of reverse transcriptase, and then the mixture was incubated at a temperature of 37° C. for 1 hour. After inactivating an enzyme for 5 minutes at a temperature of 85° C., 90 μl of RNase-free water was added to a reagent, and 100 μl of synthesized cDNA was stored at a temperature of −20° C. for future use. For qPCR of miRNA, the cDNA was combined with a miRNA-specific forward primer, mRQ 3′ reverse primer, and TB Green premix. The amplification protocol was designed to implement 40 cycles of an initial denaturation stage at a temperature of 95° C. for 10 seconds, followed by 95° C. for 5 seconds, and 60° C. for 20 seconds, and then a final melt curve stage.

During qPCR analysis for circRNA, a SuperScript IV First-Strand Synthesis System was used with 50 μM random hexamers to denature a template RNA. 11 μl of EV-derived circRNA obtained from the samples using a zeolite-NH₂-HH combination was coupled with reaction buffer and reverse transcriptase, and 20 μl of the combined reaction mixture was incubated at a temperature of 23° C. for 10 minutes, followed by 50° C. for 10 minutes, and 80° C. for 10 minutes. Then, 30 μl of RNase-free water was added to the synthesized cDNA and stored at a temperature of −20° C. until use. For qPCR analysis of circRNA, 5 μl of synthesized cDNA and a primer set were mixed with a Brilliant III SYBR Green QPCR Master Mix. The amplification protocol was designed to implement 50 cycles of an initial denaturation stage at a temperature of 94° C. for 2 minutes, followed by 94° C. for 15 seconds, 55° C. for 30 seconds, and 68° C. for 1 minute. Amplification in the protocol was finalized with a final melt curve stage.

All primers used in the qPCR are shown in Table 1 below.

TABLE 1
Primer sets for detection of CRC-related EV-derived
non-coding RNAs (miRNAs and circRNAs)

| | | | | Length |
|---|---|---|---|---|
| | Target | Location | Sequence (5′-3′) | (bp) |
| Micro | hsa-miR-19a-3p | Forward | TGT GCA AAT CTA TGC AAA ACT GA | 23 |
| RNAS | hsa-miR-21-5p | Forward | TAG CTT ATC AGA CTG ATG TTG A | 22 |
| | hsa-miR-23a-3p | Forward | ATC ACA TTG CCA GGG ATT TCC | 21 |
| | hsa-miR-92a-3p | Forward | TAT TGC ACT TGT CCC GGC CTG T | 22 |
| | hsn-mik-996-5p | Forward | CAC CCG TAG AAC CGA CCT TGC G | 22 |
| | hsa-miR-125a-3p | Forward | ACA GGT GAG GTT CTT GGG AGC C | 22 |
| | hsa-mik-141-3p | Forward | TAA CAC TGT CTG GTA AAG ATG G | 22 |
| | hsa-mik-150-5p | Forward | TCT CCC AAC CCT TGT ACCAGT G | 22 |
| | hva-miR-182-5p | Forward | TTT GGC AAT GGT AGA ACT CAC ACT | 24 |
| | hxa-miR-200h-3p | Forward | TAA TAC TGC CTG GTA ATG ATG A | 22 |
| | ksa-mik-223-3p | Forward | TGT CAG TTT GTC AAA TAC CCC A | 22 |
| | hsa-miR-1246 | Forward | AAT GGA TTT TTG GAG CAG G | 19 |
| | Universal | Reverse | mRQ 3′ Primer from TAKARA | — |
| | U6 | Forward | CTC GCT TCG GGA GCA CA | 17 |
| | | Reverse | AAC GCT TCA CGA ATT TGC GT | 20 |
| Circular | circLPAR1 | Forward | GTA GTT CTG GGG CGT GTT CA | 20 |
| RNAs | | Reverse | TAG GTG GAT GGG GAG CTT CA | 20 |
| | circPACRGL | Forward | GCC AGA AAA TAC TGA TGT TCA CT | 23 |
| | | Reverse | TCG GTC GGC CAA CAA CC | 17 |
| | circLONP2 | Forward | GTG AAG GTG GCA GAA GGA CA | 20 |
| | | Reverse | TGG GTT GTT CAC TCC CAC AG | 20 |
| | circERBIN | Forward | ACC AGC ATC CAT TGC AAA CC | 20 |
| | | Reverse | TGG TAC CAA CCG CAC AAA CA | 20 |
| | circPNN | Forward | CCT GGA AGA ATG TGT CCA GCT A | 22 |
| | | Reverse | GCT TTC TCT CTT CTT CTG CCT G | 22 |
| | GAPDH | Forward | TAT CGT GAT GCT AGT CCG ATG | 23 |
| | | Reverse | TGC AGC TAG CTG CAT CGA TCG G | 22 |

First, twelve miRNAs known to be related to the diagnosis and prognosis of CRC (miR-19, miR-21, miR-23, miR-92, miR-99, miR-125, miR-141, miR-150, miR-182, miR-200, miR-223, and miR-1246) and a housekeeping gene U6 which was a commonly used miRNA were detected. FIG. 8A shows that the zeolite-NH₂-HH combination served to successfully extract EV-derived non-coding RNAs and detect all potential CRC miRNA markers.

In addition, primers for qPCR of GAPdh, which was commonly used as circRNA and circRNA housekeeping gene, were synthesized by selecting CRC-related circRNA markers (CircLPAR1, CircPACRGL, CircLONP2, CircERBIN, and CircPNN) and using the CircInteractome database and NCBI primer design tool. Similar to EV-derived miRNAs, it was confirmed that the zeolite-NH₂-HH combination could serve to extract EV-derived circRNAs and that all five biomarkers were detected (FIG. 8B).

These results suggested that the zeolite-NH₂-HH combination of the present disclosure had the potential to serve as a one-stop platform to extract nucleic acids from EVs and that the zeolite-NH₂-HH combination might serve as a valuable tool for sample preparation in CRC diagnosis and prognosis monitoring.

### Example 9

### Clinical Validation of Selected EV-Derived Biomarkers in Blood Plasma

To confirm the clinical relevance of biomarkers identified through cell line models, blood plasma samples from CRC patients (n=80) and healthy control (HC) individuals (n=20) were analyzed using the zeolite-NH₂-HH-based extracellular vesicle isolation (ZAHVIS) platform (FIG. 9). The present inventors developed the ZAHV-AI system by combining the ZAHVIS platform with AI-driven analysis for enhanced CRC diagnosis. The ZAHVIS platform allows for simple, rapid, and cost-effective EV isolation and one-step extraction of EV-derived proteins and nucleic acids, providing a streamlined approach. The clinical characteristics of CRC patients were analyzed to determine their disease-free survival probabilities over 100 months. The experimental approach is schematically represented in FIG. 9a. This process involved the extraction of EV-derived RNAs from the blood plasma samples, followed by real-time PCR quantification of the miRNAs and circRNAs identified from cell line models, as well as control genes.

The survival rates were observed to be 95% for early-stage CRC, 75% for advanced-stage CRC, 100% for CRC stages 0-1, 90% for CRC stage 2, 80% for CRC stage 3, and 70% for CRC stage 4. The data were then subjected to statistical analysis to determine the RQ values and their significance. The present inventors focused on four statistically significant miRNAs, including miR-23a-3p, miR-92a-3p, miR-125a-3p, and miR-150-5p (FIGS. 9b-e). These miRNAs were selected from an initial pool of 7 miRNAs and 3 circRNAs based on their statistical significance in both cell line and clinical validations.

The results showed significant differences in the expression levels of miR-23a-3p, miR-92a-3p, miR-125a-3p, and miR-150-5p between CRC patients and HC individuals, indicating their potential as diagnostic biomarkers for CRC. The present inventors also measured carcinoembryonic antigen (CEA) levels and found them to be significantly higher in CRC patients compared to HC individuals, confirming CEA's established role as a diagnostic marker for CRC (FIG. 9f).

To visualize the separation between CRC patients and HC individuals based on the combined biomarker profiles, the present inventors performed t-SNE analysis (FIG. 9g). The t-SNE plot demonstrates that while some clustering occurs, there is still considerable overlap between CRC samples and HC samples. This significant overlap indicates that using these five biomarkers alone may not provide sufficient accuracy for distinguishing CRC patients from HC individuals, highlighting the need for more sophisticated analysis techniques to enhance diagnostic precision. To evaluate the diagnostic performance of each biomarker, the present inventors generated ROC curves and calculated the AUC values. While each single marker showed statistical significance, their AUC values revealed limitations in their diagnostic performance as clinical markers. The ROC curves demonstrated that miR-23a-3p had an AUC of 0.71 (fair), miR-92a-3p had an AUC of 0.77 (fair), miR-125a-3p had an AUC of 0.66 (poor), miR-150-5p had an AUC of 0.87 (good), and CEA had an AUC of 0.75 (fair) (FIG. 9h). Notably, miR-150-5p exhibited the highest AUC within the range of 0.8-0.9, suggesting it is the most promising single biomarker for CRC diagnosis, though none exceeded an AUC of 0.9 (excellent classification).

Performance evaluation metrics, including sensitivity, specificity, accuracy, and F1 score, were calculated for each biomarker according to Youden's index. miR-23a-3p showed a sensitivity of 75%, specificity of 60%, accuracy of 72%, and F1 score of 81.08%. miR-92a-3p had a sensitivity of 76.25%, specificity of 70%, accuracy of 75%, and F1 score of 82.99%. miR-125a-3p displayed a sensitivity of 35%, specificity of 100%, accuracy of 48%, and F1 score of 51.85%. miR-150-5p demonstrated the highest performance with a sensitivity of 76.25%, specificity of 90%, accuracy of 79%, and F1 score of 85.31%.

CEA exhibited a sensitivity of 66.25%, specificity of 90%, accuracy of 71%, and F1 score of 78.52% (FIG. 9i). This indicates that using these five biomarkers alone may not provide sufficient accuracy to reliably distinguish CRC patients from HC individuals. Even the highest performing single biomarker, miR-150-5p, exhibits limitations in diagnostic efficiency, underscoring the necessity for more sophisticated analysis techniques to identify the most effective biomarker combinations. These findings demonstrate the ZAHVIS platform's effectiveness in isolating and analyzing EV-derived NAs, confirming the identified miRNAs as promising CRC biomarkers.

### Example 10

### AI-Driven Comprehensive Evaluation of Blood Biomarker Combinations for Enhanced CRC Diagnosis

The comprehensive AI-driven analysis workflow for evaluating blood biomarker combinations for CRC diagnosis using the ZAHV-AI system is illustrated in FIG. 10a. Using EV-derived RNAs extracted from 100 blood plasma samples (80 CRC patients and 20 HC individuals) through the ZAHVIS platform, four miRNAs (miR-23a-3p, miR-92a-3p, miR-125a-3p, and miR-150-5p) along with the CEA marker were quantified. For each miRNA, RQ values were calculated from Ct values of three PCR repetitions for the target markers and control gene, averaged over nine RQ values per miRNA. The CEA levels were used directly, leading to 31 different biomarker combinations for AI-driven analysis. The dataset was divided into training (70%, n=70) and test (30%, n=30) sets using an optimized splitting method. Hyperparameter optimization was performed using K-fold (K=5) cross-validation. Final model parameters were selected based on the highest combined metric of (1-average validation loss) and average AUC, resulting in a learning rate of 0.1, batch size of 16, dense layer size of 128, and dropout rate of 0.1. This setup achieved the lowest average validation loss of 0.2383 and a high average AUC of 0.9432 among all tested parameter combinations. The final deep learning model comprised Z-score normalization, one input layer, two hidden layers (the first with 128 neurons and a dropout rate of 0.1, and the second with 64 neurons and a dropout rate of 0.1), and one output layer. Following model training, the performance of test set was evaluated using multiple metrics (ROC curve, AUC value, sensitivity, specificity, accuracy, and F1 score), and the optimal combinations for CRC diagnosis were identified.

The AI-driven evaluation provided critical insights across various stages of the disease. In overall CRC, single markers showed varied effectiveness, with miR-150-5p achieving the highest AUC of 0.9167. However, combinations significantly improved performance, particularly with miR-23a-3p and miR-150-5p (AUC: 0.9444), miR-125a-3p, miR-150-5p, and CEA (AUC: 0.9653), and miR-23a-3p, miR-92a-3p, miR-150-5p, and CEA (AUC: 0.9861), demonstrating the highest diagnostic accuracy. The combination of miR-23a-3p, miR-92a-3p, miR-150-5p, and CEA achieved sensitivity of 95.83%, specificity of 100%, accuracy of 96.67%, and an F1 score of 97.87% (FIG. 10). For subgroup analysis, early-stage and advanced-stage samples from the dataset were further analyzed, resulting in training (70%, n=42) and test (30%, n=18) sets. In early-stage and advanced-stage CRC, the highest single-marker AUC values were observed with miR-150-5p, with values of 0.9028 for early-stage and 0.9444 for advanced-stage. For early-stage CRC, combinations such as miR-92a-3p and miR-150-5p (AUC: 0.9722) showed improved diagnostic performance, with six combinations achieving the highest AUC of 0.9861 and high-performance metrics. For advanced-stage CRC, the combination of miR-150-5p and CEA achieved the highest AUC of 0.9583 and high-performance metrics. In stage-specific analysis, individual stage samples from the dataset were further analyzed, resulting in training (70%, n=28) and test (30%, n=12) sets. Stage-specific analysis identified several optimal combinations for each stage of CRC. For stages 0-1, three optimal combinations were identified, all achieving an AUC of 1.0 and perfect performance metrics. For stage 2, four optimal combinations were identified, all achieving an AUC of 0.9722 and high-performance metrics. For stage 3, the optimal combination of miR-150-5p and CEA was identified, achieving an AUC of 1.0 and perfect performance metrics. For stage 4, eleven optimal combinations were identified, all achieving an AUC of 1.0 and perfect performance metrics. These results show the enhanced diagnostic performance achieved through AI-driven analysis of biomarker combinations.

By leveraging multiple biomarkers, the ZAHV-AI system demonstrated significantly improved sensitivity, specificity, accuracy, and F1 scores across various stages of CRC compared to single markers. This approach enhances the diagnostic accuracy for overall CRC and shows remarkable strength in early detection, which is crucial for effective clinical management.

### Example 11

### Diagnostic Performance of Optimal Blood Biomarker Combinations for Early Detection of CRC in the ZAHV-AI System

The schemes of optimal combinations for overall CRC, early-stage CRC (stages 0-2), and advanced-stage CRC (stages 3-4) are illustrated (FIG. 11a). Diagnostic performance metrics, including AUC, sensitivity, specificity, accuracy, and F1 score, for overall CRC, early-stage CRC, advanced-stage CRC, and individual CRC stages 0-1, 2, 3, and 4 are provided (FIG. 11b). For overall CRC compared to HC, the combination of miR-23a-3p, miR-92a-3p, miR-150-5p, and CEA achieved outstanding diagnostic performance with an AUC of 0.9861, sensitivity of 95.83%, specificity of 100%, accuracy of 96.67%, and F1 score of 97.87%.

In early-stage CRC, six combinations were identified: miR-23a-3p+miR-150-5p+CEA; miR-92a-3p+miR-125a-3p+miR-150-5p; miR-92a-3p+miR-150-5p+CEA; miR-125a-3p+miR-150-5p+CEA; miR-23a-3p+miR-92a-3p+miR-150-5p+CEA; and miR-23a-3p+miR-125a-3p+miR-150-5p+CEA. These combinations yielded an AUC of 0.9861, sensitivity of 91.67%, specificity of 100%, accuracy of 94.44%, and F1 score of 95.65%. For advanced-stage CRC, the combination of miR-150-5p and CEA achieved an AUC of 0.9583, sensitivity of 91.67%, specificity of 100%, accuracy of 94.44%, and F1 score of 95.65%, highlighting the system's effectiveness in detecting both early and advanced stages of the disease. Optimal combinations specific to individual CRC stages are detailed, which includes the schemes for CRC stages 0-1, 2, 3, and 4. Detailed analysis of these stages showed high levels of diagnostic performance. For stages 0-1, perfect diagnostic performance (AUC 1.0, sensitivity 100%, specificity 100%, accuracy 100%, F1 score 100%) was achieved with three combinations: miR-150-5p; miR-92a-3p +miR-150-5p; and miR-92a-3p+miR-150-5p+CEA. Stage 2 exhibited high performance with four combinations: miR-23a-3p+miR-92a-3p; miR-92a-3p+miR-150-5p; miR-125a-3p+miR-150-5p; and miR-92a-3p+miR-125a-3p +CEA, achieving an AUC of 0.9722, sensitivity of 83.33%, specificity of 100%, accuracy of 91.67%, and F1 score of 90.91%. For stage 3, perfect diagnostic metrics were observed with the combination of miR-150-5p and CEA. Stage 4 also demonstrated perfect diagnostic performance with eleven combinations: miR-150-5p; miR-23a-3p+miR-150-5p; miR-125a-3p+CEA; miR-150-5p +CEA; miR-23a-3p+miR-125a-3p+miR-150-5p; miR-92a-3p+miR-150-5p+CEA; miR-125a-3p+miR-150-5p+CEA; miR-23a-3p+miR-92a-3p+miR-125a-3p+miR-150-5p; miR-23a-3p+miR-125a-3p+miR-150-5p+CEA; miR-92a-3p+miR-125a-3p+miR-150-5p+CEA; and miR-23a-3p+miR-92a-3p+miR-125a-3p+miR-150-5p+CEA.

These comprehensive analyses underscore the ZAHV-AI system's exceptional capability to identify optimal blood biomarker combinations, significantly enhancing diagnostic accuracy for CRC, particularly in its early stages. By integrating the efficient EV isolation using the ZAHVIS platform with AI-driven analysis, the ZAHV-AI system offers a streamlined, highly sensitive, and accurate approach to CRC diagnostics. This powerful combination not only improves early detection rates but also provides a robust tool for ongoing monitoring and personalized treatment strategies. Overall, the ZAHV-AI system demonstrates its potential for use in clinical practices, offering significant improvements in cancer diagnostics and patient care.

What is claimed is:

1. A composition for isolating an extracellular vesicle (EV) from a biological sample, the composition comprising a homobifunctional hydrazide and a solid support functionalized with an amine group (NH₂).

2. The composition of claim 1, wherein the solid support comprises one or more types selected from the group consisting of zeolite, silica gel, and diatomaceous earth.

3. The composition of claim 1, wherein the homobifunctional hydrazide has a structure of Formula I or II: 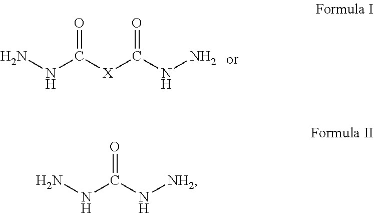
wherein in Formula I, X is (CH₂)ₙ, and n is an integer of 0 to 10.

4. The composition of claim 3, wherein n is an integer of 0 to 4.

5. The composition of claim 1, wherein the homobifunctional hydrazide comprises one or more types selected from the group consisting of an adipic acid dihydrazide, a malonic dihydrazide, an oxalyl dihydrazide, a succinic dihydrazide, and a carbonic dihydrazide that respectively have the following structures:

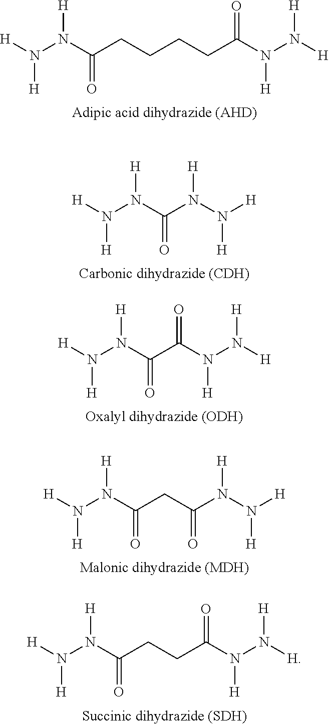

6. The composition of claim 1, wherein zeolite-NH₂ is added at a concentration of 1 to 10 mg/ml per 1 ml of the biological sample.

7. The composition of claim 1, wherein zeolite-NH₂ is added at a concentration of 2.5 to 5 mg/ml per 1 ml of the biological sample.

8. The composition of claim 1, wherein the homobifunctional hydrazide is added at a concentration of 10 to 100 mg/ml per 1 ml of the biological sample.

9. The composition of claim 1, wherein the homobifunctional hydrazide is added at a concentration of 25 to 50 mg/ml per 1 ml of the biological sample.

10. The composition of claim 1, wherein the composition is used in conjunction with a syringe filter.

11. The composition of claim 10, wherein the syringe filter has a pore size in a range of 0.22 to 3 μm.

12. A method of isolating an extracellular vesicle (EV) from a biological sample by using the composition of any one of claims 1 to 9.

13. A method of isolating an extracellular vesicle (EV) from a biological sample, the method comprising:
(a) adding the composition of any one of claims 1 to 9 to the biological sample containing the extracellular vesicle (EV);
(b) transferring the resulting mixture obtained through the (a) adding into a syringe and connecting a syringe filter to the syringe;
(c) pressing the syringe to remove an unreacted material and enriching the extracellular vesicle (EV) bound to zeolite-NH₂ on the surface of the syringe filter;
(d) isolating the extracellular vesicle (EV) from the zeolite-NH₂ by injecting a high pH elution buffer; and
(e) recovering the isolated extracellular vesicle (EV).

14. The method of claim 13, further comprising:
incubating the mixture for EV capture after the (a) adding.

15. The method of claim 13, further comprising:
washing the extracellular vesicle (EV) enriched on the surface of the syringe filter by injecting phosphate-buffered saline (PBS), after the (c) pressing.

16. The method of claim 13, wherein the syringe filter used in the (b) transferring has a pore size in a range of 0.22 to 3 μm.

17. The method of claim 13, wherein, in the (d) isolating, when a RIPA lysis buffer is injected instead of the high pH elution buffer, an EV-derived protein is extracted.

18. The method of claim 13, wherein, in the (d) isolating, when (i) a non-chaotropic lysis buffer is injected instead of the high pH elution buffer, and then (ii) a high pH elution buffer is injected, an EV-derived nucleic acid is extracted.

19. The method of claim 18, wherein the non-chaotropic lysis buffer is an NP-40 lysis buffer.
